(19) United States
(12) Patent Application Publication
(10) Pub. No.: US 2026/0285981 A1
(43) Pub. Date: Sep. 24, 2026

(54) CTLA-4-BASED LYSOSOMAL DEGRADERS AND USES THEREOF

(71) Applicant: SANOFI, Paris (FR)

(72) Inventors: Emily CONDIFF, Cambridge, MA (US); Kaori MUKAI, Cambridge, MA (US); Sunghae PARK, Cambridge, MA (US); Björn NIEBEL, Gent (BE); Victor Zhao SUN, Cambridge, MA (US); Qun ZHOU, Cambridge, MA (US)

(21) Appl. No.: 19/539,374

(22) Filed: Feb. 13, 2026

Related U.S. Application Data
(63) Continuation of application No. PCT/IB2024/058185, filed on Aug. 22, 2024.
(60) Provisional application No. 63/674,517, filed on Jul. 23, 2024.

(30) Foreign Application Priority Data
Aug. 23, 2023 (EP) 23192970.4

(51) Int. Cl. C07K 16/28; C07K 16/24

(52) U.S. Cl. CPC C07K 16/2818; C07K 16/241; C07K 2317/31; C07K 2317/622

(57) ABSTRACT

The present disclosure provides a method for degrading a target protein, comprising: contacting a T-cell with a multispecific binding protein, wherein the multispecific binding protein comprises: a) a first cell surface binding moiety that specifically binds to membrane-bound CTLA-4 on the surface of the T-cell; and b) a second binding moiety that is operatively linked to the first cell surface binding moiety and that specifically binds to the target protein, wherein binding of the multispecific binding protein to the membrane-bound CTLA-4 on the surface of the T-cell facilitates the internalization of the target protein by the T-cell. After internalization, the multispecific binding protein plus the target protein traffic to a lysosome within the T-cell, enabling degradation of the target protein within the lysosome. The present disclosure also provides multispecific binding protein compositions, host cells for making the multispecific binding protein compositions, and methods of using the multispecific binding protein compositions to treat disease.

### RELATED APPLICATIONS

This application is a continuation of International Patent Application No. PCT/IB2024/058185, filed Aug. 22, 2024, which claims priority U.S. Patent Application Ser. No. 63/674,517, filed Jul. 23, 2024, and European Patent Application No. 23192970.4, filed Aug. 23, 2023, the entire disclosures of which are hereby incorporated herein.

### SEQUENCE LISTING

The instant application contains a Sequence Listing which has been submitted electronically in XML format and is hereby incorporated by reference in its entirety. Said XML file, created on Feb. 12, 2026, is named 756931_SA9-376PCCON_ST26.xml, and is 55,965 bytes in size.

### FIELD OF THE INVENTION

The subject disclosure relates to novel multispecific binding proteins or binding fragments thereof, that bind to CTLA-4 and a target protein of interest. Methods of using the binding proteins to facilitate degradation of the target protein of interest are also provided.

### BACKGROUND

Over the past two decades target protein degradation technologies have elicited great interest in expanding the landscape of druggable targets. It has also provided a unique mechanism of action for therapeutics as “event-driven” pharmacology as opposed to “occupancy-driven” associated with conventional inhibitors. For example, PROteolysis TArgeting Chimeras or PROTACs, small heterobifunctional molecules that form a ternary complex with an E3 ubiquitin ligase and a target of interest, resulting in target ubiquitination and degradation, have advanced through clinical trials. However, the therapeutic potential of PROTACs has been hampered by the poor permeability, pharmacokinetics, and pharmacodynamic properties commonly seen with high molecular mass small molecules (over 1,000 Da). More recently, large molecule-based degrader technologies, such as lysosome targeting chimeras (LYTACs), have highlighted the potential of leveraging large molecules for targeted degradation of extracellular soluble and membrane-associated proteins. There is a need in the art for targeted degradation strategies, particularly those that are able to degrade targets in specific cell types.

### BRIEF SUMMARY OF THE INVENTION

The present disclosure is based on the surprising discovery that the endogenous CTLA-4 lysosomal shuttling pathway of a T-cell can be co-opted to degrade target molecules through T-cell specific, CTLA-4-mediated lysosomal degradation. The subject specification provides multispecific binding proteins which are capable of binding CTLA-4 (e.g., membrane-bound CTLA-4) and a target protein of interest. In certain aspects, binding of membrane bound CTLA-4 by the multispecific binding protein facilitates endocytosis and shuttling of the target protein to the lysosomal compartment of the T-cell for subsequent degradation.

In certain aspects, the disclosure provides a method for degrading a target protein, comprising: contacting a T-cell with a multispecific binding protein, wherein the multispecific binding protein comprises: a) a first cell surface binding moiety that specifically binds to membrane-bound CTLA-4 on the surface of the T-cell; and b) a second binding moiety that is operatively linked to the first cell surface binding moiety and that specifically binds to the target protein, wherein binding of the multispecific binding protein to the membrane-bound CTLA-4 on the surface of the T-cell facilitates the internalization of the target protein by the T-cell.

In certain aspects, after internalization of the target protein it is degraded in a lysosome. In certain aspects, the multispecific binding protein exhibits increased degradation of the target protein compared to a reference binding polypeptide.

In certain aspects, the first cell surface binding moiety that specifically binds to membrane-bound CTLA-4 on the surface of the T-cell comprises the variable domain of a CTLA-4 antibody or an antigen binding fragment thereof. In an aspect, the first cell surface binding moiety that specifically binds to membrane-bound CTLA-4 on the surface of the T-cell comprises a CTLA-4 binding portion of a CTLA-4 ligand. In certain aspects, the CTLA-4 ligand is selected from the group consisting of CD80 and CD86. In certain aspects, the CTLA-4 ligand is the extracellular domain of CD80 or CD86. In certain aspects, the first cell surface binding moiety comprises a CD80-fragment crystallizable (Fc) fusion polypeptide or a CD86-Fc fusion polypeptide.

In certain aspects, the target protein is selected from the group consisting of an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

In certain aspects, the target protein is a tumor secreted protein. In certain aspects, the target protein is expressed on the T-cell membrane. In an aspect, the T-cell is an activated T cell or a regulatory T (Treg) cell. In certain aspects, the target protein is an immune checkpoint protein. In certain aspects, the target protein is associated with a disease selected from the group consisting of a cancer, an autoimmune disease, an inflammatory disorder, an infectious disease, and a neurodegenerative disorder. In certain aspects, the target protein is associated with the cancer. In certain aspects, the target protein is associated with the autoimmune disease. In certain aspects, the target protein is associated with the inflammatory disorder.

In certain aspects, the first cell surface binding moiety of the multispecific binding protein that specifically binds to membrane-bound CTLA-4 on the surface of the T-cell is an antibody or an antigen binding fragment thereof, an Fc fusion protein, a fragment antigen-binding (Fab) fusion, an immunoglobulin single variable domain (ISV), or a single-chain fragment variable (scFv).

In certain aspects, the first cell surface binding moiety of the multispecific binding protein that specifically binds to membrane-bound CTLA-4 on the surface of the T-cell is an antibody or an antigen binding fragment thereof.

In certain aspects, the first cell surface binding moiety of the multispecific binding protein that specifically binds to membrane-bound CTLA-4 on the surface of the T-cell is the Fc fusion protein, wherein the Fc fusion protein comprises a CTLA-4 ligand-Fc fusion protein.

In certain aspects, the first cell surface binding moiety of the multispecific binding protein that specifically binds to membrane-bound CTLA-4 on the surface of the T-cell is the scFv, wherein the scFv is a linear scFv or a tandem scFv.

In certain aspects, the first cell surface binding moiety of the multispecific binding protein that specifically binds to membrane-bound CTLA-4 on the surface of the T-cell is the ISV.

In certain aspects, the ISV is a V_{HH}, humanized V_{HH} or camelized V_{H}.

In certain aspects, the second binding moiety of the multispecific binding protein that specifically binds to the target protein is selected from the group consisting of an antigen, an antibody or an antigen binding fragment thereof, an autoantibody, a Fc fusion protein, a Fab, an scFv, an ISV, an AFFIBODY®, or a peptide.

In certain aspects, the second binding moiety of the multispecific binding protein that specifically binds to the target protein is the antibody or an antigen binding fragment thereof.

In certain aspects, the second binding moiety of the multispecific binding protein that specifically binds to the target protein is the ISV. In certain aspects, the ISV is a V_{HH}, a humanized V_{HH} or a camelized V_{H}.

In certain aspects, the first cell surface binding moiety of the multispecific binding protein exhibits pH-dependent binding to membrane-bound CTLA-4 on the surface of the T-cell. In certain aspects, the second binding moiety of the multispecific binding protein exhibits pH-dependent binding to the target protein. In certain aspects, the multispecific binding protein exhibits reduced binding at acidic pH.

In certain aspects, the first cell surface binding moiety of the multispecific binding protein binds to membrane-bound CTLA-4 on the surface of the T-cell with an affinity from about 100 pM to about 1 μM. In certain aspects, the second binding moiety of the multispecific binding protein binds to the target protein with an affinity from about 100 pM to about 1 μM.

In certain aspects, the multispecific binding protein comprises one or more mutations or glycan modifications to modulate the Fc mediated effector function.

In certain aspects, the multispecific binding protein comprises one or more mutations to modulate half-life.

In one aspect, the disclosure provides a multispecific binding protein comprising: a) a first cell surface binding moiety that specifically binds to membrane-bound CTLA-4 on the surface of a T-cell; and b) a second binding moiety that is operatively linked to the first cell surface binding moiety and that specifically binds to a target protein, such that the multispecific binding protein binds to the membrane-bound CTLA-4 on the surface of the T-cell and to the target protein, wherein binding to the membrane-bound CTLA-4 on the surface of the T-cell facilitates the internalization of the target protein by the T-cell.

In certain aspects, after internalization the target protein is degraded in a lysosome. In certain aspects, the multispecific binding protein exhibits increased degradation of the target protein compared to a reference binding polypeptide.

In certain aspects, the first cell surface binding moiety that specifically binds to membrane-bound CTLA-4 on the surface of the T-cell is a CTLA-4 ligand. In certain aspects, the CTLA-4 ligand is selected from the group consisting of CD80 and CD86.

In certain aspects, the target protein is selected from the group consisting of an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

In certain aspects, the target protein is a tumor secreted protein. In certain aspects, wherein the target protein is expressed on a T-cell membrane. In certain aspects, the T-cell is an activated T cell or a Treg cell. In certain aspects, the target protein is an immune checkpoint protein. In certain aspects, the target protein is associated with a disease selected from the group consisting of cancer, autoimmune diseases, inflammatory disorders, infectious diseases, and neurodegenerative disorders. In certain aspects, the target protein is associated with cancer. In certain aspects, the target protein is associated with autoimmune diseases. In certain aspects, the target protein is associated with inflammatory disorders.

In certain aspects, the first cell surface binding moiety of the multispecific binding protein that specifically binds to membrane-bound CTLA-4 on the surface of the T-cell is an antibody or an antigen binding fragment thereof, an Fc fusion protein, a Fab fusion, an ISV, or a scFv.

In certain aspects, the first cell surface binding moiety that specifically binds to membrane-bound CTLA-4 on the surface of the T-cell is the antibody or an antigen binding fragment thereof.

In certain aspects, the first cell surface binding moiety that specifically binds to membrane-bound CTLA-4 on the surface of the T-cell is the Fc fusion protein, wherein the Fc fusion protein comprises a CTLA-4 ligand-Fc fusion protein.

In certain aspects, the first cell surface binding moiety that specifically binds to membrane-bound CTLA-4 on the surface of the T-cell is the scFv, wherein the scFv is a linear scFv or a tandem scFv. In certain aspects, the first cell surface binding moiety of the multispecific binding protein that specifically binds to membrane-bound CTLA-4 on the surface of the T-cell is the ISV. In certain aspects, the ISV is a V_{HH}, humanized V_{HH} or camelized V_{H}.

In certain aspects, the second binding moiety that specifically binds to the target protein is selected from the group consisting of an antigen, an antibody or an antigen binding fragment thereof, an autoantibody, a Fc fusion, a Fab, an scFv, an ISV, an AFFIBODY®, or a peptide. In certain aspects, the second binding moiety that specifically binds to the target protein is the ISV. In certain aspects, the ISV is a V_{HH}, a humanized V_{HH} or a camelized V_{H}.

In certain aspects, the first cell surface binding moiety of the multispecific binding protein exhibits pH-dependent binding to a membrane-bound CTLA-4 on the surface of the T-cell. In certain aspects, the second binding moiety of the multispecific binding protein exhibits pH-dependent binding to the target protein. In certain aspects, the multispecific binding protein further comprises a modification that allows pH-dependent binding of the multispecific binding protein to the membrane-bound CTLA-4 on the surface of the T-cell. In certain aspects, the modification reduces binding at acidic pH.

In certain aspects, the first cell surface binding moiety of the multispecific binding protein binds to the membrane-bound CTLA-4 on the surface of the T-cell with an affinity from about 100 pM to about 1 μM. In certain aspects, the second binding moiety of the multispecific binding protein binds to the target protein with an affinity from about 100 pM to about 1 μM.

In certain aspects, the multispecific binding protein further comprises one or more mutations or glycan modifications to modulate the Fc mediated effector function.

In certain aspects, the multispecific binding protein further comprises one or more mutations to modulate half-life.

In one aspect, the disclosure provides a pharmaceutical composition comprising any of the multispecific binding proteins disclosed herein. In another aspect, the composition further comprises a pharmaceutically acceptable carrier.

In one aspect, the disclosure provides an isolated nucleic acid molecule encoding any of the multispecific binding proteins disclosed herein. In certain aspects, the disclosure provides an expression vector comprising the nucleic acid molecule as disclosed herein. In certain aspects, the disclosure provides a host cell comprising the expression vector as disclosed herein.

In one aspect, the disclosure provides a method of treating a subject with a disease associated with the target protein or soluble target protein, comprising administering to the subject a therapeutically effective amount of any of the multispecific binding proteins described herein or any of the pharmaceutical compositions disclosed herein.

In one aspect, the disclosure provides a method of treating a subject wherein binding of the multispecific binding protein to the membrane-bound CTLA-4 on the surface of the T-cell facilitates the internalization and the trafficking of the target protein by the T-cell to a lysosome within the T-cell, enabling degradation of the target protein within the lysosome, thereby treating the disease in the subject.

In certain aspects, the disease is selected from the group consisting of a cancer, an autoimmune disease, an inflammatory disorder, an infectious disease, and a neurodegenerative disorder. In certain aspects, the disease is the cancer. In certain aspects, the disease is the autoimmune disease. In certain aspects, the disease is the inflammatory disorder.

In certain aspects, the disclosure provides a method of treating a subject wherein the multispecific binding protein is administered by intravenous, subcutaneous, intramuscular, or intradermal injection.

The summary of the disclosure described above is non-limiting and other features and advantages of the disclosed multispecific binding proteins, or binding fragments or derivatives thereof and methods will be apparent from the following brief description of the drawings, detailed description of the disclosure, and claims.

### BRIEF DESCRIPTION OF THE FIGURES

FIG. 1 displays a schematic illustration of the internalization of a target protein (e.g., TNFα) by a TNFα/CTLA-4 multispecific binding protein disclosed herein in a CTLA-4 surface expressing cell. A wild-type Raji cell line (Raji-null cells) was engineered to stably express human CTLA-4 on the cell surface membrane (Raji-CTLA-4 cells).

FIGS. 2A-2B display internalization of CTLA-4 by Raji-CTLA-4 cells an hour after the addition of an anti-CTLA-4 antibody (FIG. 2A) or after the addition of an antibody isotype control (FIG. 2B). CTLA-4 was rapidly internalized after Raji-CTLA-4 expressing cells were incubated with an anti-CTLA-4 antibody for one hour as determined by confocal microscopy (FIG. 2A).

FIGS. 3A-3B display internalization of CTLA-4 by Raji-CTLA-4 cells four hours after the addition of an anti-CTLA-4 antibody (FIG. 3A) or the of addition of an antibody isotype control (FIG. 3B).

FIG. 4 is a schematic of the bispecific antibody complex used to visualize the internalization and lysosomal trafficking steps of the target protein, TNFα, after binding to an anti-TNFα/CTLA-4 bispecific antibody construct disclosed herein. The bispecific antibody complex was comprised of a biotinylated human TNFα, streptavidin conjugated to AlexaFlour488 (streptavidin-AF488), and an anti-TNFα/CTLA-4 bispecific antibody construct described herein.

FIG. 5 are representative confocal maximum intensity projection images of Raji-CTLA-4 cells incubated for two-(left column) or four-hours (right column) with the bispecific antibody complex described in FIG. 4. After incubation cells were fixed and co-stained with fluorescently labeled endosome and lysosome markers, anti-EEA1 and anti-LAMP1 antibodies, as well as a nuclear stain (Hoechst). Scale bar: 15 μm.

FIG. 6 are representative confocal images of Raji-CTLA-4 cells incubated for one-(top), two-(middle), or four-hours (bottom) with the bispecific antibody complex described in FIG. 4. Scale bar: 20 μm.

FIG. 7 are representative confocal maximum intensity projection images of Raji-CTLA-4 cells incubated for one-(left column), two-(middle column), or four-hours (right column) with the bispecific antibody complex described in FIG. 4. After incubation, cells were fixed and co-stained using endosome and lysosome markers, rabbit anti-EEA1 and mouse anti-LAMP1 antibodies, fluorescently-labeled goat anti-rabbit and goat anti-mouse secondary antibodies, as well as a nuclear stain (Hoechst). Images were captured using ZEISS Airyscan Joint Deconvolution for optimized resolution and channel separation. Scale bar: 15 μm.

FIG. 8 are representative confocal maximum intensity projection overlay images of Raji-CTLA-4 cells incubated for one-(top), two-(middle), or four-hours (bottom) with the same experimental set-up as described in FIG. 7. Overlay images show fluorescent labeling for the bispecific antibody complex, EEA1, LAMP1, and the nucleus. Scale bar: 5 μm.

FIG. 9 is a graph of the weighted colocalization values to quantify the different levels of colocalization for EEA1 and LAMP1 fluorescent markers with the staining from the bispecific antibody complex at 1-, 2-, and 4-hour time course experiments described in FIGS. 7 and 8.

FIG. 10 shows two schematics of the bispecific antibody complex described in FIG. 4 which was further modified by either conjugation of pHrodo-red to the bispecific antibody (top) or streptavidin-AF488 (bottom).

FIG. 11 shows representative confocal maximum intensity projection overlay images of Raji-CTLA-4 cells incubated at 0, 9, 30, or 45-minutes with pHrodo-red stain and Lyso View™ stain (LysoView™ is a lysosomal marker).

FIG. 12 shows two graphs displaying the mean fluorescence intensity (MFI) of images collected during a 60-minute time-course generated by Raji-CTLA-4 cell incubation with either a modified bispecific antibody conjugated to pHrodo-streptavidin as described in FIG. 10 or pHodo-antibody complex described in FIG. 4.

FIG. 13 is a graph displaying the MFI of images collected during a 60-minute time-course generated by incubation of either Raji-null cells or Raji-CTLA-4 cells with a modified bispecific antibody conjugated to pHrodo-red.

FIGS. 14A-14B display the internalization of TNFα by a TNF/CTLA-4 bispecific antibody in Raji-CTLA4 or control cells. FIG. 14A displays CTLA-4 staining in Raji-CTLA4 cells versus control (Raji-null cells). FIG. 14B displays the internalization of TNFα by Raji-CTLA-4 cells after treatment with 25 or 100 nM of either anti-TNFα/CTLA-4 bispecific antibody construct plus a fluorescently labeled-TNFα at a concentration of at 25 and 100 nM or with an antibody isotype control plus a fluorescently labeled TNFα.

FIG. 15 displays the degradation of TNFα by an anti-TNFα/CTLA-4 bispecific antibody as determined by western blotting. Raji-CTLA-4 or Raji-null cells (control) were incubated with either the anti-TNFα/CTLA-4 bispecific antibody plus TNFα or with an antibody isotype control plus TNFα for two hours followed by a wash. Both cell sample groups were then either treated with Bafilomycin (a potent lysosomal inhibitor) or DMSO at several timepoints.

FIGS. 16A-16B display the internalization of TNFα by an anti-TNFα/CTLA-4 bispecific antibody using FACS analysis. FIG. 16A displays a representative FACS plots showing the population of CD4⁺ cells in a peripheral blood mononuclear cells (PBMC) sample. FIG. 16B displays the internalization of fluorescently labeled-TNFα in phytohaemagglutinin (PHA) activated PBMC by an anti-TNFα/CTLA-4 bispecific antibody or an antibody isotype control after incubation for four and twenty-four hours. The PHA activated PBMC were derived from two donors (“Donor 1” and “Donor 2”).

FIG. 17 displays TNFα internalization in Raji-CTLA-4 or Raji-null cells (control) upon incubation with fluorescently labeled-TNFα plus escalating concentrations using three different anti-TNFα/CTLA-4 bi-specific V_{HH} constructs. There is no TNFα internalization observed in Raji-null cells or with the V_{HH} construct controls expressing a single binding domain for either CTLA-4 or TNFα.

FIG. 18 is a graphical representation of the results of the FACs analysis performed in FIG. 17 represented in MFI.

FIG. 19 displays the fold increase of TNFα internalization in Raji-CTLA-4 cell relative to Raji-null cells (control) upon incubation with fluorescently labeled-TNFα and anti-TNFα/CTLA-4 bi-specific V_{HH} constructs or control V_{HH} constructs expressing a single binding domain for either CTLA-4 or TNFα.

FIG. 20 displays a western blot image of the degradation of TNFα in the lysosome of Raji-CTLA-4 cells upon 2-, 9-, or 24-hour incubation with TNFα plus one of the three anti-TNFα/CTLA-4 bi-specific V_{HH} constructs. As a control, Bafilomycin was added to some of the samples.

FIG. 21 displays a western blot image of the reduction of TNFα in cell culture media after TNFα (either at 12.5 nM or at 50 nM) was added to the cell culture medium of Raji-CTLA-4 cells which were either incubated for 24-hours with 25 nM or 100 nM of one of the three anti-TNFα/CTLA-4 bi-specific V_{HH} constructs or V_{HH} controls.

### DETAILED DESCRIPTION

Provided herein are compositions and methods for the degradation of target molecules through CTLA-4-mediated lysosomal degradation. The compositions and methods of the instant disclosure employ multispecific binding proteins comprising a first binding moiety that specifically interacts with CTLA-4 on T-cells (e.g., membrane bound CTLA-4) and a second binding moiety which specifically binds to the target molecule of interest (e.g., a pathogenic protein).

It is to be understood that the methods described in this disclosure are not limited to particular methods and experimental conditions such that methods and conditions can vary. It is also to be understood that the terminology used herein is for the purpose of describing particular embodiments only and is not intended to be limiting.

### Polypeptide and Isolated Polypeptide

The term “polypeptide” refers to any polymeric chain of amino acids and encompasses native or artificial proteins, polypeptide analogs or variants of a protein sequence, or fragments thereof, unless otherwise contradicted by context. A polypeptide can be monomeric or polymeric. For a polypeptide (e.g., a polypeptide encoding a first cell surface binding moiety that specifically binds to CTLA-4 on the surface of a CTLA-4 positive cell and/or a second binding moiety that is operatively linked to the first cell surface binding moiety and that specifically binds to a target protein), a fragment of a polypeptide optionally contains at least one contiguous or nonlinear epitope of a polypeptide. A fragment polypeptide can be about 25, 50, 75, 100, 150, 200, 250, 300, 350, 400 or more amino acids in length while retaining the capacity to bind to both CTLA-4 and a target protein. The precise boundaries of the at least one epitope fragment can be confirmed using ordinary skill in the art. A polypeptide fragment comprises at least about 5 contiguous amino acids, at least about 10 contiguous amino acids, at least about 15 contiguous amino acids, or at least about 20 contiguous amino acids, at least about 50 contiguous amino acids, at least about 100 contiguous amino acids, at least about 150 contiguous amino acids, at least about 200 contiguous amino acids, at least about 250 contiguous amino acids, at least about 300 contiguous amino acids, at least about 400 contiguous amino acids for example.

In certain aspects, a first cell surface binding moiety and a second binding moiety polypeptides are “isolated polypeptides.” The term “isolated polypeptide” refers to a protein or polypeptide that by virtue of its origin or source of derivation is not associated with naturally associated components that accompany it in its native state; is substantially free of other proteins from the same species. An isolated recombinant polypeptide is expressed by a cell from a different species. In some aspects, an isolated polypeptide does not occur in nature. A protein or polypeptide that is chemically synthesized or synthesized in a cellular system can be different from the cell from which it naturally originates and therefore will be “isolated” from its naturally associated components. A protein or polypeptide can also be rendered substantially free of naturally associated components by isolation using protein purification techniques.

### Binding Protein or Binding Polypeptide

As used herein, the term “binding protein,” “binding polypeptide,” or “multispecific binding polypeptide or protein” refer to a protein or polypeptide (e.g., an antibody or specific binding fragment thereof a Fab, an scFv, an immunoglobulin single variable domain such as a NANOBODY® molecule, an AFFIBODY®, a V_{HH}, or a peptide) that contains at least one binding site which is responsible for selectively binding to a target antigen of interest (e.g., a human antigen). Binding sites include an antibody variable domain, a ligand binding site of a receptor, or a receptor binding site of a ligand. In certain aspects, the binding proteins or binding polypeptides comprise multiple (e.g., two, three, four, or more) binding sites. In certain aspects, the binding protein or binding polypeptide is not a therapeutic enzyme.

An affibody (or non-immunoglobulin binding scaffold protein) refers to any of a class of small (approximately 6 kDa) polypeptide antibody mimetics comprising a three alpha helix bundle domain of about 58 amino acids in length. Affibodies are derived from the immunoglobulin binding domains of staphylococcal Protein A. See, e.g., Nord et al., Protein Eng. 8:601-608 (1995). Affibodies have high stability, withstanding temperatures as high as 90° C., and have no Fc function. Affibody binding sites can be synthesized by mutagenizing a staphylococcal Protein A-related protein (e.g., Protein Z) derived from a domain of Protein A (e.g., domain B) and selecting for mutant polypeptides having binding affinity for a target. Affibody binding sites can also be produced by the methods described in U.S. Pat. Nos. 6,740,734, 6,602,977, and WO 2000/063243.

### Ligand and Antigen

The term “ligand” refers to any substance capable of binding, or of being bound, to another substance. The term “antigen” or “target antigen” as used herein refers to a molecule or a portion of a molecule that is capable of being bound by the binding site of a binding polypeptide e.g., any substance to which an antibody can be generated. A target antigen may have one or more epitopes.

Although “antigen” is commonly used in reference to an antibody binding substrate, and “ligand” is often used when referring to receptor binding substrates, these terms are not distinguishing, one from the other, and encompass a wide range of overlapping chemical entities. For the avoidance of doubt, antigen and ligand are used interchangeably throughout herein.

Examples of antigens/ligands can be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

### Immunoglobulin Domain

The term immunoglobulin domain as used herein can refer to an immunoglobulin A, an immunoglobulin D, an immunoglobulin E, an immunoglobulin G, or an immunoglobulin M. The immunoglobulin domain can be an immunoglobulin heavy chain region or a fragment thereof. In some instances, the immunoglobulin domain is from an antibody (e.g., a monoclonal antibody, a mammalian antibody, a recombinant antibody, a chimeric antibody, an engineered antibody, a human antibody, a humanized antibody) or an antigen binding fragment thereof.

### Antibody

As used herein, the term “antibody” refers to such assemblies (e.g., intact antibody molecules, antibody fragments, or variants thereof) which have significant known specific immunoreactive activity to an antigen of interest (e.g., a cell surface CTLA-4 on a T cell or a target protein associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” comprises five distinct classes of antibody that can be distinguished biochemically. While all five classes of antibodies are clearly within the scope of the current disclosure, the following discussion will generally be directed to the IgG class of immunoglobulin molecules. With regard to IgG, immunoglobulins comprise two identical light chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin isotype subclasses (e.g., IgG1, IgG2, IgG3, IgG4, IgAQ1, etc.) confer functional specialization. Modified versions of each of these classes and isotypes are readily discernable to the skilled artisan in view of the instant disclosure and, accordingly, are within the scope of the current disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs,” as defined herein.

### Constant and Variable Domains

The regions of an immunoglobulin heavy or light chain can be defined as “constant” (C) region or “variable” (V) regions, based on the relative lack of sequence variation within the regions of various class members in the case of a “constant region”, or the significant variation within the regions of various class members in the case of a “variable regions.” The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three-dimensional configurations of the constant regions of the various immunoglobulin classes are well known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains.” Constant domains on the heavy chain (e.g., hinge, CH1, CH2 or CH3 domains) are referred to interchangeably as “heavy chain constant region domains”, “CH” region domains or “CH domains”. Variable domains on the light chain are referred to interchangeably as “light chain variable region domains”, “VL region domains” or “VL domains.” Variable domains on the heavy chain are referred to interchangeably as “heavy chain variable region domains”, “VH region domains” or “VH domains.”

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat. Numbering of the heavy chain constant region is via the EU index as set forth in Kabat (Kabat, Sequences of Proteins of Immunological Interest, National Institutes of Health, Bethesda, MD, 1987 and 1991).

In some aspects, a multispecific binding protein comprises: a) a first cell surface binding moiety that specifically binds to membrane-bound CTLA-4 on the surface of a T-cell; and b) a second binding moiety that is operatively linked to the first cell surface binding moiety and that specifically binds to a target protein, such that the multispecific binding protein binds to the membrane-bound CTLA-4 on the surface of the T-cell and to the target protein, wherein binding to the membrane-bound CTLA-4 on the surface of the T-cell facilitates the internalization of the target protein by the T-cell. In some aspects, after internalization the target protein is degraded in a lysosome. In some aspects, the multispecific binding protein exhibits increased degradation of the target protein compared to a reference binding polypeptide.

In some aspects, the first cell surface binding moiety that specifically binds to membrane-bound CTLA-4 on the surface of the T-cell is a CTLA-4 ligand. In some aspects, the CTLA-4 ligand is selected from the group consisting of CD80 and CD86. In some aspects, the first cell surface binding moiety that specifically binds to membrane-bound CTLA-4 on the surface of the T-cell is the antibody or an antigen binding fragment thereof. In some aspects, the first cell surface binding moiety that specifically binds to membrane-bound CTLA-4 on the surface of the T-cell is an Fc fusion protein, wherein the Fc fusion protein comprises a CTLA-4 ligand-Fc fusion protein.

In some aspects, the second binding moiety that specifically binds to the target protein is selected from the group consisting of an antigen, an antibody or an antigen binding fragment thereof, an autoantibody, a Fc fusion, a Fab, an scFv, an ISVD, a NANOBODY®, an AFFIBODY®, or a peptide.

Without being bound by any particular mechanistic theory, upon binding membrane-bound CTLA-4, the multispecific binding protein facilitates internalization (e.g., endocytosis) of the target protein into the T-cell, initiating a series of intracellular events which culminate in shuttling or trafficking of the target protein of interest to the lysosomal compartment of the T-cell for subsequent degradation. By harnessing or co-opting the endogenous CTLA-4 shuttling pathway of the T-cell, the methods of the disclosure enable efficient and selective degradation of various target molecules (both soluble and membrane bound), such as proteins secreted by tumors, autoantibodies, inflammatory proteins, interleukins, and signaling molecules. These can be used in targeted therapies with precise control over the degradation of specific molecules, providing avenues for the treatment of diseases such as autoimmune disorders, cancer, and inflammatory conditions, resulting in a more balanced immune response, and improved clinical outcomes.

The term “cytotoxic T-lymphocyte antigen 4” or “CTLA-4” as used herein refers to a membrane-bound receptor that is a member of the immunoglobulin (Ig) superfamily that is expressed by activated T-cells. CTLA-4 is homologous to the T-cell co-stimulatory protein, CD28, and binds B7-1/CD80 and B7-2/CD86 ligands present on antigen presenting cells (APCs). CTLA-4 is also found on the surface membrane of regulatory T cells. CTLA-4 is also referred to as cytotoxic T-lymphocyte-associated protein 4, CD152, Insulin-dependent Diabetes Mellitus 12 (IDDM12), Celiac Disease 3 (CELIAC3), GRD4, and GSE. The term “CTLA-4” includes any variants or isoforms of CTLA-4 which are naturally expressed by cells.

The term “T-cell” as used herein is defined as a thymus-derived lymphocyte that participates in a variety of cell-mediated immune reactions. In certain aspects, the T-cell is a regulatory T cell. The term “regulatory T-cell” as used herein refers to a CD4+ CD25+ FoxP3+ T cell with suppressive properties. “Treg” is the abbreviation used herein for a regulatory T-cell. T-cells activated T-cells, and Treg cells can express membrane bound CTLA-4.

The term “specifically binds”, “binds specifically to”, “specific for” or the like, means that the multispecific binding protein, or a binding fragment or derivative thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind include, for example, equilibrium dialysis, surface plasmon resonance, and the like.

In some aspects, a constant domain can be a Fc domain. As used herein, the term “Fc domain” or “Fc region” (used interchangeably) is defined as the portion of a heavy chain constant region beginning in the hinge region just upstream of the papain cleavage site (i.e., residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The term “Fc variant,” “modified Fc,” “engineered Fc” interchangeably used herein refers to a molecule or sequence that is modified from a native Fc but still comprises a binding site for the salvage receptor, FcRn (neonatal Fc receptor). Exemplary Fc variants, and their interaction with the salvage receptor, are known in the art. A modified Fc domain can comprise a molecule or sequence that is humanized from a non-human native Fc. Furthermore, a native Fc comprises regions that can be removed because they provide structural features or biological activity that are not required for the conjugate compositions. Thus, the term “modified Fc domain” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

“Effector function” as used herein is meant a biochemical event that results from the interaction of an antibody Fc region with an Fc receptor or ligand. A “functional Fc region” possesses an “effector function” of a native sequence Fc region. Exemplary “effector functions” include antibody-dependent cell-mediated cytotoxicity (ADCC) or antibody-dependent cell-mediated phagocytosis (ADCP).

The term “EU index” refers to the EU numbering convention for the constant regions of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. Unless otherwise stated, all antibody Fc region numbering employed herein corresponds to the EU numbering scheme, as described in Edelman et al. (Proc. Natl. Acad. Sci. 63(1): 78-85. 1969).

Again, without being bound to any particular mechanistic theory, when a first binding moiety of a multispecific binding protein binds to CTLA-4 on a T-cell surface, this triggers receptor-mediated endocytosis and internalization of a complex comprising the CTLA-4 receptor and the multispecific binding protein bound to CTLA-4 via the first binding moiety and to the target of interest via a second binding moiety. In particular, the interaction of the first binding moiety with CTLA-4 elicits the clustering of CTLA-4 receptors, leading to the formation of endocytic vesicles. The clustering process induces the recruitment of adaptors and clathrin-coated pits to the cell membrane, initiating the process of receptor-mediated endocytosis. Following the formation of clathrin-coated pits, the clustered CTLA-4 receptors are internalized by invagination of the plasma membrane, resulting in the formation of clathrin-coated vesicles. These vesicles containing the CTLA-4 receptors pinch off from the plasma membrane and enter the cytoplasm. Once inside the cytoplasm, the clathrin-coated vesicles shed their clathrin coats, forming uncoated endocytic vesicles that transport the internalized CTLA-4 receptors to early endosomes. Early endosomes are intracellular compartments involved in sorting and trafficking of cargo molecules. Within the early endosomes, the internalized CTLA-4 receptors can undergo further sorting processes. From the early endosomes, the CTLA-4 receptors are directed to late endosomes, which are characterized by a lower pH environment due to the presence of proton pumps. The decreasing pH within the late endosomes triggers the fusion of lysosomes, specialized organelles containing various hydrolytic enzymes, with the late endosomes. This fusion event leads to the formation of endolysosomes. The target proteins or target proteins bound to the multi-specific binding protein, or a binding fragment or derivative thereof are co-internalized along with CTLA-4 into the endolysosomal compartment. The endolysosomes then fuse with the lysosome, where the target proteins are exposed to the hydrolytic enzymes present in the lysosomes. These enzymes facilitate the degradation of the target protein of interest into smaller peptides and amino acids, ultimately leading to their complete breakdown within the lysosome.

### I. Multispecific Binding Protein Formats

As used herein, a “multispecific” binding protein is a binding protein that specifically binds two or more antigens. A multispecific binding protein that binds two antigens, and/or two different epitopes of different antigens, is also referred to herein as a “bispecific” binding protein. A multispecific binding protein that binds three antigens, and/or three different epitopes, is also referred to herein as a “trispecific” binding protein. Thus, the multispecific binding protein is able to bind two or more different targets simultaneously, for example, membrane bound CTLA-4 and a target protein of interest. Genetic engineering can be used to design, modify, and produce the multispecific binding protein, or a binding fragment or derivative thereof with a desired set of binding properties and effector functions.

A multispecific binding protein of this disclosure, in certain aspects, comprises a first cell surface binding moiety that specifically binds to membrane-bound CTLA-4 on the surface of a T-cell and a second binding moiety that is operatively linked to the first cell surface binding moiety and specifically binds to a target protein.

In certain aspects, the first cell surface binding moiety of the multispecific binding protein that specifically binds to membrane-bound CTLA-4 on the surface of the T-cell is an antibody or an antigen binding fragment thereof. In certain aspects, the first cell surface binding moiety of the multispecific binding protein that specifically binds to membrane-bound CTLA-4 on the surface of the T-cell is the Fc fusion protein, wherein the Fc fusion protein comprises a CTLA-4 ligand-Fc fusion protein.

In certain aspects, the first cell surface binding moiety and the second binding moiety of the multispecific binding protein, is each independently selected from a group consisting of an antibody or an antigen-binding fragment thereof (such as, scFv, Fab, Fab′, Fv, F(ab′)₂), a minibody, a diabody, a triabody, a tetrabody, a tandem di-scFv, a tandem tri-scFv, an immunoglobulin single variable domain (ISV), such as, a NANOBODY®, a V_{HH} (including humanized V_{HH}), a camelized V_{H}, a single domain antibody, a domain antibody, or a dAb).

In certain aspects, the first cell surface binding moiety of the multispecific binding protein that specifically binds to membrane-bound CTLA-4 on the surface of the T-cell is an antibody or an antigen binding fragment thereof, an Fc fusion protein, a fragment antigen-binding (Fab) fusion, an immunoglobulin single variable domain (ISV), or a single-chain fragment variable (scFv). In certain aspects, the first cell surface binding moiety of the multispecific binding protein that specifically binds to membrane-bound CTLA-4 on the surface of the T-cell is the scFv, wherein the scFv is a linear scFv or a tandem scFv. In certain aspects, the first cell surface binding moiety of the multispecific binding protein that specifically binds to membrane-bound CTLA-4 on the surface of the T-cell is the ISV. In certain aspects, the ISV is a V_{HH}, humanized V_{HH} or camelized V_{H}.

In certain aspects, the second binding moiety of the multispecific binding protein that specifically binds to the target protein is selected from the group consisting of an antigen, an antibody or an antigen binding fragment thereof, an autoantibody, a Fc fusion protein, a Fab, an scFv, an ISV, an AFFIBODY®, or a peptide. The second binding moiety of the multispecific binding protein that specifically binds to the target protein can be the ISV. In certain aspects, the ISV is a V_{HH}, a humanized V_{HH} or a camelized V_{H}.

ISVs have advantages over conventional antibodies: they are about ten times smaller than IgG molecules, and as a consequence properly folded functional ISVs can be produced by in vitro expression while achieving high yield. Furthermore, ISVs are very stable, and resistant to the action of proteases. The properties and production of ISVs have been reviewed by Harmsen and De Haard H J (Appl. Microbiol. Biotechnol. 2007 November; 77(1):13-22).

In certain aspects, the first cell surface binding moiety of the multispecific binding protein is an antibody and the second binding moiety of the multispecific binding protein that specifically binds to the target protein is an antibody. In certain aspects, the first cell surface binding moiety of the multispecific binding protein is an ISV and the second binding moiety of the multispecific binding protein that specifically binds to the target protein is an ISV.

In certain aspects, the first cell surface binding moiety of the multispecific binding protein is an antibody and the second binding moiety of the multispecific binding protein that specifically binds to the target protein is an ISV. In certain aspects, the first cell surface binding moiety of the multispecific binding protein is an ISV and the second binding moiety of the multispecific binding protein that specifically binds to the target protein is an antibody. In certain aspects, the first cell surface binding moiety of the multispecific binding protein is an antibody fragment and the second binding moiety of the multispecific binding protein that specifically binds to the target protein is an antibody. In other aspects, the first cell surface binding moiety of the multispecific binding protein is an antibody and the second binding moiety of the multispecific binding protein that specifically binds to the target protein is an antibody fragment. In certain aspects, the first cell surface binding moiety of the multispecific binding protein is an antibody fragment, and the second binding moiety of the multispecific binding protein that specifically binds to the target protein is an antibody fragment. In other aspects, the first cell surface binding moiety of the multispecific binding protein is an ISV and the second binding moiety of the multispecific binding protein that specifically binds to the target protein is an antibody fragment. In other aspects, the first cell surface binding moiety of the multispecific binding protein is an antibody fragment and the second binding moiety of the multispecific binding protein that specifically binds to the target protein is an ISV. In other aspects, the first cell surface binding moiety of the multispecific binding protein is an ISV (e.g., a V_{HH}) and the second binding moiety of the multispecific binding protein that specifically binds to the target protein is an ISV (e.g., a V_{HH}).

The terms “binding moiety” and “binding domain” may be used interchangeably herein. A binding moiety of the multispecific binding protein can be an antibody, a Fab fragment, a F(ab′)₂ fragment, an Fv fragment, an immunoglobulin single variable domain (ISV, such as a V_{HH}), an scFv fragment, a fragment containing a complementarity determining region (CDR), an isolated CDR, or other suitable fragment.

In certain aspects, the term “antigen binding fragment” refers to a polypeptide fragment of a multispecific binding protein. Antigen-binding fragments of a multispecific binding protein, or a binding fragment or derivative thereof can be derived, e.g., from full multispecific binding protein molecules using any suitable standard techniques such as proteolytic digestion or recombinant genetic engineering techniques involving the manipulation and expression of DNA encoding multispecific binding protein, or a binding fragment or derivative thereof variable and (optionally) constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add, or delete amino acids, etc.

As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

A “CDR” or “complementarity determining region,” or individual specified CDRs (e.g., “HCDR1,” “HCDR2,” “HCDR3”), of a given antibody or region thereof, such as a variable region thereof, should be understood to encompass a (or the specific) complementarity determining region as defined by any of the known schemes. Likewise, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the IMGT, Kabat, Chothia, AbM, or Contact method. In other cases, the particular amino acid sequence of a CDR or FR is given. Unless otherwise specified, all particular CDR amino acid sequences mentioned in the disclosure are IMGT CDRs. However, alternative CDRs defined by other schemes are also encompassed by the present disclosure, such as those determined by abYsis Key Annotation (Website: abysis.org/abysis/sequence_input/key_annotation/key_annotation.cgi).

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. Other engineered molecules, such as domain-specific binding proteins, single domain binding proteins, domain-deleted binding proteins, chimeric binding proteins, CDR-grafted binding proteins, diabodies, triabodies, tetrabodies, minibodies, immunoglobulin single variable domains (ISVs) (e.g., monovalent ISVs, bivalent ISVs, etc.), small modular immunopharmaceuticals (SMIPs), and shark variable IgNAR domains, are also encompassed within the expression “antigen-binding fragment,” as used herein. The term “multispecific antibody” denotes a binding fragment or derivative thereof that combines the antigen-binding sites of two or more antibodies within a single molecule. The terms “antigen-binding portion”, “antigen-binding fragment”, “binding protein” or “binding moiety” and the like, as used herein, include any naturally occurring, enzymatically obtainable, synthetic, or genetically engineered polypeptide or glycoprotein that specifically binds to at least one target antigen to form a complex. In certain aspects, a binding moiety can refer to one or more fragments of a multispecific binding protein that retains the ability to specifically bind to membrane bound CTLA-4 on the surface of the T-cell and/or a second target protein or a target protein.

In some aspects, the antigen-binding fragments of the disclosure are immunoglobulin single variable domains (ISVs), such as a domain antibody, a “dAb”, a V_{HH} (including a humanized V_{HH}), a camelized V_{H}, other single variable domains, or any suitable fragment of any one thereof. In particular, antigen-binding fragments of the disclosure may be a V_{HH} or a fragment thereof.

The term “immunoglobulin single variable domain” (ISV or ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation.

ISVs of the so-called “V_{H}3 class” (i.e., ISVs with a high degree of sequence homology to human germline sequences of the V_{H}3 class such as DP-47, DP-51 or DP-29), can be used herein. Furthermore, any type of ISV directed against membrane-bound CTLA-4 on the surface of the T-cell and/or target protein, including for example, ISVs belonging to the so-called “V_{H}4 class” (i.e., ISVs with a high degree of sequence homology to human germline sequences of the V_{H}4 class such as DP-78), as for example described in WO 2007/118 670 A1.

ISVs (in particular V_{HH} sequences and partially humanized V_{HH}s) can in particular be characterized by the presence of one or more “Hallmark residues” (as described herein in Table 1 and in subsequent paragraphs describing NANOBODY® immunoglobulin single variable domains) such that the ISV is a NANOBODY® ISV.

Thus, generally, a NANOBODY® ISV (in particular a V_{HH}, including (partially or fully) humanized V_{HH} and camelized V_{H}) can be defined as an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which one or more of the Hallmark residues are as further defined in Table 1. In particular, a NANOBODY® ISV (in particular a V_{HH}, including (partially) humanized V_{HH} and camelized V_{H}) can be an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which the framework sequences are as further defined herein. More in particular, an ISV can be an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively.

ISVs can specifically bind to (as defined herein) and/or are directed against membrane-bound CTLA-4 on the surface of the T-cell and/or the target protein. Also useful are suitable fragments of these ISVs and polypeptides that comprise or essentially consist of one or more of such ISVs and/or suitable fragments of the ISVs. The term “immunoglobulin single variable domain (ISV)” encompasses a NANOBODY® V_{HH} as described in or WO 08/020079 or WO 09/138519, and thus in an aspect denotes a V_{HH}, a humanized V_{HH} or a camelized V_{H} (such as a camelized human V_{H}) or generally a sequence optimized V_{HH} (such as e.g., optimized for chemical stability and/or solubility, maximum overlap with known human framework regions and maximum expression).

Generally, NANOBODY® immunoglobulin single variable domains (ISVs) (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISV can be defined as an immunoglobulin sequence with the (general) structure

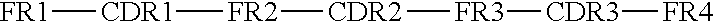

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a NANOBODY® ISV can be an immunoglobulin sequence with the (general) structure:

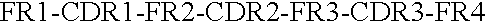

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a NANOBODY® ISV can be an immunoglobulin sequence with the (general) structure:

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in Nanobody ® ISVs.

| Position | Human V_{H}3 | Hallmark Residues |
|---|---|---|
| 11 | L, V; | L, S, V, M, W, F, T, Q, E, A, R, G, K, Y, N, P, I; preferably L |
| | predominantly L | |
| 37 | V, I, F; | F⁽¹⁾, Y, V, L, A, H, S, I, W, C, N, G, D, T, P, preferably F⁽¹⁾ or Y |
| | usually V | |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾. |
| 45⁽⁸⁾ | L | L⁽²⁾, R⁽³⁾, P, H, F, G, Q, S, E, T, Y, C, I, D, V; preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾, L⁽¹⁾ or F⁽¹⁾ |
| 83 | R or K; | R, K⁽⁵⁾, T, E⁽⁵⁾, Q, N, S, I, V, G, M, L, A, D, Y, H; preferably |
| | usually R | K or R; most preferably K |
| 84 | A, T, D; | P⁽⁵⁾, S, H, L, A, V, I, T, F, D, R, Y, N, Q, G, E; preferably P |
| | predominantly A | |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; | Q, L⁽⁷⁾, R, P, E, K, S, T, M, A, H; preferably Q or L⁽⁷⁾ |
| | predominantly L | |

Notes:
⁽¹⁾In particular, but not exclusively, in combination with KERE (SEQ ID NO: 12) or KQRE (SEQ ID NO: 13) at positions 43-46.
⁽²⁾Usually as GLEW (SEQ ID NO: 47) at positions 44-47.
⁽³⁾Usually as KERE (SEQ ID NO: 12) or KQRE (SEQ ID NO: 13) at positions 43-46, e.g. as KEREL (SEQ ID NO: 14), KEREF(SEQ ID NO: 15), KQREL(SEQ ID NO: 16), KQREF(SEQ ID NO: 17), KEREG (SEQ ID NO: 18), KQREW(SEQ ID NO: 19) or KQREG(SEQ ID NO: 20) at positions 43-47. Alternatively, also sequences such as TERE(SEQ ID NO: 21)(for example TEREL(SEQ ID NO: 22)), TQRE(SEQ ID NO: 23) (for example TQREL(SEQ ID NO: 24)), KECE (SEQ ID NO: 25) (for example KECEL(SEQ ID NO: 26) or KECER(SEQ ID NO: 27)), KQCE(SEQ ID NO: 28) (for example KQCEL(SEQ ID NO: 29)), RERE(SEQ ID NO: 30) (for example REREG(SEQ ID NO: 31)), RQRE (SEQ ID NO: 58) (for example RQREL(SEQ ID NO: 32), RQREF(SEQ ID NO: 33) or RQREW(SEQ ID NO: 34)), QERE (SEQ ID NO: 35)(for example QEREG(SEQ ID NO: 36)), QQRE(SEQ ID NO: 37), (for example QQREW(SEQ ID NO: 38), QQREL(SEQ ID NO: 39) or QQREF(SEQ ID NO: 40)), KGRE(SEQ ID NO: 41) (for example KGREG(SEQ ID NO: 42)), KDRE(SEQ ID NO: 43) (for example KDREV(SEQ ID NO: 44)) are possible. Some other possible, but less preferred sequences include for example DECKL(SEQ ID NO: 45) and NVCEL.(SEQ ID NO: 46)
⁽⁴⁾With both GLEW (SEQ ID NO: 47) at positions 44-47 and KERE (SEQ ID NO: 12) or KQRE (SEQ ID NO: 13) at positions 43-46.
⁽⁵⁾Often as KP or EP at positions 83-84 of naturally occurring V_{HH} domains.
⁽⁶⁾In particular, but not exclusively, in combination with GLEW (SEQ ID NO: 47) at positions 44-47.
⁽⁷⁾With the proviso that when positions 44-47 are GLEW (SEQ ID NO: 47), position 108 is always Q in (non-humanized) V_{HH} sequences that also contain a W at 103.
⁽⁸⁾The GLEW (SEQ ID NO: 47) group also contains GLEW (SEQ ID NO: 47) -like sequences at positions 44-47, such as for example GVEW(SEQ ID NO: 48), EPEW(SEQ ID NO: 49), GLER(SEQ ID NO: 50), DQEW(SEQ ID NO: 51), DLEW(SEQ ID NO: 52), GIEW(SEQ ID NO: 53), ELEW(SEQ ID NO: 54), GPEW(SEQ ID NO: 55), EWLP(SEQ ID NO: 56), GPER(SEQ ID NO: 57), GLER(SEQ ID NO: 50) and ELEW(SEQ ID NO: 54).

In certain aspects, the first cell surface binding moiety and the second binding moiety, of the multispecific binding protein, each independently comprises an antigen-binding fragment that comprises at least one variable domain of an antibody or an antigen binding fragment covalently linked to at least one constant domain. The variable domain can be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains can be situated relative to one another in any suitable arrangement. For example, the variable region can be dimeric and contain V_{H}—V_{H}, V_{H}-V_{L} or V_{L}-V_{L} dimers. Alternatively, the antigen-binding fragment can contain a monomeric V_{H} or V_{L} domain.

Non-limiting, exemplary configurations of variable and constant domains that can be found within an antigen-binding fragment include: (i) V_{H}-CH1; (ii) V_{H}-CH2; (iii) V_{H}-CH3; (iv) V_{H}-CH1-CH2; (v) V_{H}—CH1-CH2-CH3; (vi) V_{H}-CH2-CH3; (vii) V_{H}-CL; (viii) V_{L}-CH1; (ix) V_{L}-CH2; (X) V_{L}-CH3; (xi) V_{L}-CH1-CH2; (xii) V_{L}-CH1-CH2-CH3; (xiii) V_{L}-CH2-CH3; and (xiv) V_{L}-CL. In any configuration of variable and constant domains, including any of the exemplary configurations listed above, the variable and constant domains can be either directly linked to one another or can be linked by a full or partial hinge or linker region. A hinge region can comprise of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of a multispecific binding protein can comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In some aspects, the first cell surface binding moiety and the second binding moiety, of the multispecific binding protein, is each independently an scFv. A single chain Fv (“scFv”) polypeptide is a covalently linked V_{H}:V_{L} heterodimer that is usually expressed from a gene fusion including V_{H} and V_{L} encoding genes linked by a peptide-encoding linker. A human scFv fragment includes CDRs that are held in appropriate conformation by, e.g., using gene recombination techniques. Divalent and multivalent multi-specific binding proteins, or binding fragments or derivatives thereof can form either spontaneously by association of monovalent scFvs, or can be generated by coupling monovalent scFvs by a peptide linker, such as divalent sc(Fv)₂. A “dsFv” is a V_{H}:V_{L} heterodimer stabilized by a disulfide bond. “(dsFv)₂” denotes two dsFv coupled by a peptide linker.

In certain aspects, the first cell surface binding moiety and the second binding moiety, of the multispecific binding protein, is each independently a Fab. The term “Fab” denotes a binding protein or a binding fragment thereof having a molecular weight of about 50,000 Da and antigen binding activity, in which about a half of the N-terminal side of H chain and the entire L chain, among fragments obtained by treating IgG with a protease, papain, are bound together through a disulfide bond.

In certain aspects, the first cell surface binding moiety and the second binding moiety, of the multispecific binding protein, is each independently a Fab″ or F(ab″)₂. The term “F(ab″)₂” refers to a binding protein or a binding fragment thereof having a molecular weight of about 100,000 Da and antigen binding activity, which is slightly larger than a Fab bound via a disulfide bond of the hinge region, among fragments obtained by treating IgG with a protease, pepsin. The term Fab″ refers to a binding protein or a binding fragment having a molecular weight of about 50,000 Da and antigen binding activity, which is obtained by cutting a disulfide bond of the hinge region of the F(ab″)₂.

In certain aspects, the first cell surface binding moiety and the second binding moiety, of the multispecific binding protein, is each independently an immunoglobulin single variable domain (ISV). Examples of immunoglobulin single variable domains (ISVs) include variable domains obtained from heavy chain antibodies (V_{HH}s), variable domains obtained from antibodies naturally devoid of light chains (V_{HH}s), ISVs derived from conventional four-chain antibodies, engineered ISVs. ISVs may be derived from any species including, but not limited to mouse, human, camel, llama, goat, rabbit, bovine. ISVs may be naturally occurring ISVs present in a heavy chain antibody devoid of light chains. In particular, Camelidae species, for example camel, dromedary, llama, alpaca and guanaco, produce heavy chain antibodies naturally devoid of light chain. Camelid heavy chain antibodies also lack the CH1 domain.

In certain exemplary aspects, methods of the disclosure comprise contacting a T-cell with a bispecific ISV construct, wherein the bispecific ISV construct comprises: a) a first ISV that specifically binds to membrane-bound CTLA-4 on the surface of the T-cell; and b) a second ISV that specifically binds to a target protein of interest, such that the bispecific ISV construct binds to the membrane-bound CTLA-4 on the surface of the T-cell and to the target protein.

In certain exemplary aspects, methods of the disclosure comprise contacting a T-cell with a bispecific NANOBODY® ISV (such as a V_{HH}, including a humanized V_{H} or a camelized V_{H}), wherein the bispecific NANOBODY® ISV comprises: a) a first NANOBODY® ISV (such as a V_{HH}, including a humanized V_{H} or a camelized V_{H}) that specifically binds to membrane-bound CTLA-4 on the surface of the T-cell; and b) a second NANOBODY® ISV (such as a V_{HH}, including a humanized V_{H} or a camelized V_{H}) that specifically binds to the target protein of interest, such that the bispecific NANOBODY® ISV binds to the membrane-bound CTLA-4 on the surface of the T-cell and to the target protein.

Techniques for making multispecific binding proteins (e.g., multispecific antibodies) include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). Multispecific antibodies may also be made by engineering electrostatic steering effects for making binding protein Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making multispecific binding protein fragments (see, e.g., Holliger, P. et al., Proc. Natl. Acad. Sci. USA 90 (1993) 6444-6448); and using single-chain Fv (scFv) dimers (see, e.g., Gruber, M et al., J. Immunol. 152 (1994) 5368-5374); and preparing trispecific binding proteins as described, e.g., in Tutt, A. et al., J. Immunol. 147 (1991) 60-69).

A wide variety of recombinant multispecific binding protein formats have been developed, e.g., by fusion of, e.g. an IgG binding protein format and single chain domains (see Kontermann R E, mAbs 4:2, (2012) 1-16). Multispecific binding proteins wherein the variable domains V_{L} and V_{H} or the constant domains CL and CH1 are replaced by each other are described in WO2009080251 and WO2009080252.

An approach to circumvent the problem of mispaired byproducts, which is known as “knobs-into-holes”, aims at forcing the pairing of two different binding protein heavy chains by introducing mutations into the CH3 domains to modify the contact interface. On one chain bulky amino acids were replaced by amino acids with short side chains to create a “hole”. Conversely, amino acids with large side chains were introduced into the other CH3 domain, to create a “knob”. By co-expressing these two heavy chains (and two identical light chains, which have to be appropriate for both heavy chains), high yields of heterodimer formation (“knob-hole”) versus homodimer formation (“hole-hole” or “knob-knob”) was observed (Ridgway J B, Presta L G, Carter P; and WO1996027011). The percentage of heterodimer could be further increased by remodeling the interaction surfaces of the two CH3 domains using a phage display approach and the introduction of a disulfide bridge to stabilize the heterodimers (Merchant A. M, et al, Nature Biotech 16 (1998) 677-681; Atwell S, Ridgway J B, Wells J A, Carter P., J Mol Biol 270 (1997) 26-35). New approaches for the knobs-into-holes technology are described in e.g. in EP 1870459A1. Xie, Z., et al, J Immunol Methods 286 (2005) 95-101 refers to a format of multispecific binding protein using scFvs in combination with knobs-into-holes technology for the Fc part.

In certain aspects, the CH3 domains of the heavy chains of the multispecific binding protein are altered by the “knob-into-holes” technology which is described in detail with several examples in e.g., WO 96/027011, WO 98/050431, Ridgway J. B. et al., Protein Eng. 9 (1996) 617-621, Merchant A. M. et al., Nat Biotechnol 16 (1998) 677-681. In this method, the interaction surfaces of the two CH3 domains are altered to increase the heterodimerization of both heavy chains containing said two CH3 domains. Each of the two CH3 domains (of the two heavy chains) can be the “knob,” while the other is the “hole.” The introduction of a disulfide bridge can be utilized to stabilize the heterodimers (Merchant A. M et al., Nature Biotech 16 (1998) 677-681, Atwell, S. et al., J. Mol. Biol. 270 (1997) 26-35), as well as to increase the yield.

The Fc domain of a bispecific antibody may be engineered to promote heterodimerization over homodimerization. For example, the heavy chain constant region of the first heavy-light chain pair may comprise a different amino acid sequence from the heavy chain constant region of the second heavy-light chain pair, wherein the different amino acid sequences are engineered to promote heterodimerization of the heavy chain constant regions. Examples include knobs-into-holes mutations or charge pair mutations. Alternatively, the heavy chain constant region of the first heavy-light chain pair may be identical to the heavy chain constant region of the second heavy-light chain pair, in which case it is expected that both homodimers and heterodimers will assemble, and these will be subsequently separated using one or more purification steps in the antibody manufacturing process to isolate the desired heterodimer comprising one anti-CTLA-4 arm and one anti-target protein arm.

Multispecific binding proteins can be provided in various isotypes and with different constant regions. The Fc region of antibodies is recognized by Fc receptors and determines the ability of the antibody to mediate cellular effector functions, including antibody-dependent cell-mediated cytotoxicity (ADCC) activity, complement dependent cytotoxicity (CDC) activity and antibody-dependent cell phagocytosis (ADCP) activity. These cellular effector functions involve recruitment of cells bearing Fc receptors to the site of the target cells, resulting in killing of the antibody-bound cell.

### II. CTLA-4 Binding Moieties

In certain aspects, the multispecific binding proteins of the disclosure comprise a binding domain or moiety that binds CTLA-4 (e.g., human CTLA-4) to facilitate lysosomal targeting. In certain aspects, the CTLA-4 is an endogenous cell membrane-bound surface receptor expressed on the surface of T-cells. In certain aspects, a cell membrane-bound surface receptor is CTLA-4 expressed on the surface membrane of a T-cell.

Exemplary CTLA-4 binding moieties can be derived from CTLA-4 antibodies that are obtained by immunizing mice with native CTLA-4 or a full length recombinant CTLA-4 peptide. Alternatively, CTLA-4 or a fragment thereof can be produced using biochemical techniques and modified and used as immunogen. In certain aspects, an immunogen can be a peptide from the N terminal or C terminal end of CTLA-4. In some aspects, an immunogen can be a recombinant CTLA-4 peptide expressed in a prokaryote, such as E. coli, or in eukaryotic cells or mammalian cells such as Chinese hamster ovary (CHO) cells. In other aspects, the extracellular domain of human CTLA-4 is used to raise the CTLA-4 antibody. In certain aspects, the CTLA-4 antibody can be obtained by immunizing a transgenic mouse that expresses the human immune repertoire (e.g., the VELOCIMMUNE® mouse from Regeneron). The VELOCIMMUNE® mouse comprises a genome comprising human heavy and light chain variable regions operably linked to endogenous mouse constant region loci such that the mouse produces antibodies comprising a human variable region and a mouse constant region in response to antigenic stimulation. The DNA encoding the variable regions of the heavy and light chains the CTLA-4 antibody can be isolated and incorporated into the multispecific binding proteins of the disclosure.

In certain aspects, the CTLA-4 binding moiety comprises the variable domains of a CTLA-4 antibody known in the art. For example, the multispecific binding protein of the disclosure can comprise a CTLA-4 binding moiety comprising the amino acid sequences of a known anti-CTLA-4 binding protein, e.g., Ipilimumab or Tremelimumab. In other aspects, the CTLA-4 binding protein is the bioequivalent of a known binding protein. For the bioequivalent CTLA-4 binding protein can comprise amino acid sequences that vary from those of known CTLA-4 binding proteins, but that retain the ability to bind CTLA-4. Such variant CTLA-4 binding proteins comprise one or more additions, deletions, or substitutions of amino acids when compared to a parent sequence but exhibit biological activity that is essentially equivalent to that of the known CLTA-4 binding protein. Two CTLA-4 binding proteins are considered bioequivalent if, for example, they are pharmaceutical equivalents or pharmaceutical alternatives whose rate and extent of absorption do not show a significant difference when administered at the same molar dose under similar experimental conditions, either as a single dose or multiple doses. Some CTLA-4 binding proteins will be considered equivalents or pharmaceutical alternatives if they are equivalent in the extent of their absorption but not in their rate of absorption and yet can be considered bioequivalent because such differences in the rate of absorption are intentional and are reflected in the labeling, are not essential to the attainment of effective body drug concentrations on, e.g., chronic use, and are considered medically insignificant for the particular drug product studied. Bioequivalent variants of known CTLA-4 antibodies can be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent CTLA-4 binding moieties can include variants comprising amino acid changes, which modify the glycosylation characteristics of known CTLA-4 binding proteins e.g., mutations that eliminate or remove glycosylation.

In some aspects, the first cell surface binding moiety that specifically binds to membrane-bound CTLA-4 on the surface of the T-cell comprises the variable domain of a CTLA-4 antibody or an antigen binding fragment thereof. In some aspects, the first cell surface binding moiety that specifically binds to membrane-bound CTLA-4 on the surface of the T-cell comprises a CTLA-4 binding portion of a CTLA-4 ligand. In some aspects, the CTLA-4 ligand is selected from the group consisting of CD80 and CD86. In some aspects, the CTLA-4 ligand is the extracellular domain of CD80 or CD86. In some aspects, the first cell surface binding moiety comprises a CD80-fragment crystallizable (Fc) fusion polypeptide or a CD86-Fc fusion polypeptide.

In some aspects, the CTLA-4 binding moiety comprises a ligand of CTLA-4 (e.g., CD80 or CD86) or CTLA-4 binding portion thereof. In certain aspects, the first cell surface target binding moiety is a fusion of the CTLA-4 ligand and an Fc domain (e.g., a CD80-Fc fusion polypeptide or a CD86-Fc fusion polypeptide). In certain aspects, the Fc domain can be engineered to pair or heterodimerize with the second binding moiety which binds the target molecule of interest. For example, the multispecific binding protein of the disclosure may comprise a first polypeptide comprising a fusion of a CTLA-4 ligand and an Fc domain and a second polypeptide comprising a binding specificity (e.g., a V_{HH}, Fab or scFv) for a target protein of interest, wherein the binding specificity is fused to a second Fc domain capable of dimerizing with the first Fc domain. Accordingly, binding of the multispecific binding protein to membrane-bound CTLA-4 via the CTLA-4 ligand and dimerization of the first and second polypeptides can facilitate internalization and degradation of the target protein of interest.

In general, multispecific binding proteins as described herein can function by binding to membrane bound CTLA-4 and a target protein with high affinity or avidity. Dimerization of CTLA-4 promotes its endocytosis through clathrin-coated pits, resulting in enhanced internalization and lysosomal degradation. In certain aspects, the multispecific binding protein can bind membrane bound CTLA-4 and/or a soluble target protein with a K_{D} of less than about 1 μM as measured by surface plasmon resonance (e.g., at 25° C. or at 37° C.). In certain aspects, the multispecific binding protein bind CTLA-4 and/or the target protein with a K_{D} of less than about 40 nM, less than about 30 nM, less than about 20 nM, less than about 10 nM less than about 5 nM, less than about 2 nM or less than about 1 nM, as measured by surface plasmon resonance.

In certain aspects, the multispecific binding proteins described herein bind CTLA-4 with a dissociative half-life (t½) of greater than about 1.1 minutes as measured by surface plasmon resonance at, e.g., about 25° C. or 37° C. In certain aspects, the multispecific binding proteins bind CTLA-4 and the soluble target protein with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C.

In certain aspects, the multispecific binding proteins described herein comprise a modified binding moiety to alter binding affinity compared to a conjugate comprising a wild-type binding moiety. In some aspects, a modified binding moiety has an enhanced binding affinity compared to a conjugate comprising a wild-type binding moiety. In some aspects, a binding moiety is modified to enhance the binding affinity at an acidic pH compared to a conjugate comprising a wild-type binding moiety. In some aspects, a binding moiety is modified to enhance the binding affinity at a basic pH compared to a conjugate comprising a wild-type binding moiety. In some aspects, a modified binding moiety has a decreased binding affinity compared to a conjugate comprising a wild-type binding moiety. In some aspects, a binding moiety is modified to decrease the binding affinity at an acidic pH compared to a conjugate comprising a wild-type binding moiety. In some aspects, a binding moiety is modified to decrease the binding affinity at a basic pH compared to a conjugate comprising a wild-type binding moiety. In certain aspects, the first cell surface binding moiety of the multispecific binding protein binds to the membrane-bound CTLA-4 on the surface of the T-cell with an affinity from about 100 pM to about 1 μM (e.g., about 100 pM to about 1,000 μM, about 1,000 pM to about 0.01 μM, about 0.01 μM to about 0.1 μM, or about 0.1 μM to about 1.0 μM). In other aspects, the second binding moiety of the multispecific binding protein binds to the target protein with an affinity from about 100 pM to about 1 μM (e.g., about 100 pM to about 1,000 pM, about 1,000 pM to about 0.01 μM, about 0.01 μM to about 0.1 μM, or about 0.1 μM to about 1.0 μM).

In some aspects, the first cell surface binding moiety of the multispecific binding protein binds to membrane-bound CTLA-4 on the surface of the T-cell with an affinity from about 100 pM to about 1 μM. In some aspects, the second binding moiety of the multispecific binding protein binds to the target protein with an affinity from about 100 pM to about 1 μM.

### III. pH Sensitive CTLA-4 Binding

In certain aspects, the multispecific binding proteins of the disclosure comprise a CTLA-4 binding moiety which exhibits pH-sensitive binding to CTLA-4. In certain aspects, the pH-sensitive binding moiety facilitates dissociation from CTLA-4 within the lysosomal compartment, allowing CTLA-4 and/or the multispecific binding protein to recycle back to the cell surface where it can bind additional target protein for degradation. For example, the CTLA-4 binding moiety may comprise a Fab domain comprising one or more mutations which enhance or diminish binding to CTLA-4 under different pH conditions e.g., at acidic pH as compared to neutral pH. For example, the CTLA-4 binding portion of the multispecific antibody can comprise a mutation in the CH1, CL, V_{H}, or the V_{L} region of the Fab domain, wherein the mutation(s) increases the affinity of the Fab domain to its antigen in an acidic environment (e.g., in tumor microenvironment where pH is about 7.2, 7.0, 6.8, 6.5, 6.3 or lower. Such mutations can result in an increase in serum half-life of the multispecific binding protein when administered to an animal.

In certain aspects, the sensitivity of CTLA-4 binding at acidic pH may be increased, whereby the anti-CTLA-4 binding moiety demonstrates reduced binding to CTLA-4 at lower pH. In one aspect, binding to CTLA-4 is reduced at a pH that reflects the endosomal compartment. In certain aspects, binding to CTLA-4 is reduced at pH 5.5 relative to binding at neutral pH (pH 7.0). Such reduced binding at pH 5.5 may be 50% or more of the CTLA-4 binding observed at neutral pH. A change, such as a reduction or increase, in an anti-CTLA-4 activity described herein, such as binding, may be in comparison to a reference or wild-type antibody. The reference antibody may be an antibody known in the art such as Ipilimumab or Tremelimumab. The change may also be relative between two different pH levels of a particular antibody composition described herein. pH sensitive anti-CTLA-4 antibodies may be identified by testing the interaction between plate coated CTLA-4 and soluble CTLA-4 antibodies over a pH range of 4.5 to 7.0, and selecting antibodies with increased pH sensitivity such that reduced binding is observed at acidic pH. Examples of an anti-CTLA-4 antibody with reduced binding to CTLA-4 at acidic pH comprises replacing tyrosine with histidine within or near one or more CDR1-3 regions of at least one of a light chain and heavy chain variable region of the antibody. See WO2020214748A1, incorporated by reference in its entirety.

In certain aspects, the first cell surface binding moiety of the multispecific binding protein exhibits pH-dependent binding to membrane-bound CTLA-4 on the surface of the T-cell. In certain aspects, the second binding moiety of the multispecific binding protein exhibits pH-dependent binding to the target protein. In certain aspects, the multispecific binding protein exhibits reduced binding at acidic pH. In certain aspects, the first cell surface binding moiety of the multispecific binding protein binds to membrane-bound CTLA-4 on the surface of the T-cell with an affinity from about 100 pM to about 1 μM.

### IV. Species Selectivity and Species Cross-Reactivity

In certain aspects, the multispecific binding proteins of the disclosure employ CTLA binding moieties which bind to human CTLA-4 but not to CTLA-4 from other species. Alternatively, the multispecific binding protein employ a CTLA-4 binding moiety which binds to human CTLA-4 and to CTLA-4 from one or more non-human species. For example, the multispecific binding protein can bind to human CTLA-4 and can bind or not bind, as the case can be, to one or more of mouse, rat, guinea pig, hamster, gerbil, pig, cat, dog, rabbit, goat, sheep, cow, horse, camel, cynomolgus, marmoset, rhesus or chimpanzee CTLA-4. In certain aspects, the multispecific binding protein can bind to human CTLA-4, but do not bind to rat and mouse CTLA-4. In other aspects, the multispecific binding protein bind to human CTLA-4, and to rat and mouse CTLA-4, with similar binding affinities.

### V. Fc Mutations to Silence or Enhance Effector Function and to Increase the Half-Life

In certain aspects, the multispecific binding proteins employ CTLA-4 targeting agents which are capable of selectively depleting or stimulating a target protein, e.g., in the tumor microenvironment. In one aspect, the anti-CTLA-4 binding moieties have increased or decreased Fc mediated activity. In certain aspects, target protein depletion can occur by Fc mediated effector function such as antibody-dependent cell-mediated cytotoxicity (ADCC) or antibody-dependent cell-mediated phagocytosis (ADCP). In another aspect, the multispecific binding proteins comprise Fc domain variants in which at least one amino acid in one or more of the constant region domains has been deleted or otherwise altered so as to provide desired biochemical characteristics such as reduced or enhanced effector functions, the ability to non-covalently dimerize, increased ability to localize at the site of a tumor, reduced serum half-life, increased serum half-life, enhanced binding affinities at acidic pH, reduced binding affinities at acidic pH, enhanced binding affinities at non-acidic pH, reduced binding affinities at non-acidic pH, when compared with a whole, unaltered antibody of approximately the same immunogenicity.

The Fc mediated effector function of the CTLA-4 binding moiety can be introduced, enhanced, or silenced by any method known in the art. For example, the multispecific binding protein can comprise an Fc portion with enhanced effector function. In certain aspects, Fc enhancing mutations (S298A, E333A and K334A) can be introduced into the CH region of the Fc domain to increase ADCC activity. Additionally or alternatively, the Fc portion can be afucosylated to increase antibody-dependent cellular cytotoxicity (ADCC). For example, Biowa's POTELLIGENT® technology uses a FUT8 gene knockout CHO cell line to produce 100% afucosylated antibodies. FUT8 is the only gene coding al, 6-Fucosyltransferase which catalyzes the transfer of Fucose from GDP-Fucose to GlcNAc in al, 6-linkage of complex-type oligosaccharide. Probiogen has developed a CHO line that is engineered to produce lower levels of fucosylated glycans on MAbs, although not through FUT knockout. Probiogen's system introduces a bacterial enzyme that redirects the de-novo fucose synthesis pathway towards a sugar-nucleotide that cannot be metabolized by the cell. As an alternative approach, Seattle Genetics has a proprietary feed system which will produce lower levels of fucosylated glycans on MAbs produced in CHO (and perhaps other) cell lines. Xencor has developed an XmAb Fc domain technology is designed to improve the immune system's elimination of tumor and other pathologic cells. This Fc domain has two amino acid changes, resulting in a 40-fold greater affinity for FcγRIIIa. It also increases affinity for FcγRIIa, with potential for recruitment of other effector cells such as macrophages, which play a role in immunity by engulfing and digesting foreign material (see WO2019152423A1).

In certain aspects, the Fc region of the anti-CTLA-4 binding moieties of the multispecific binding proteins of the current disclosure may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, WO06/085967A2, and WO21/016571A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated in its entirety by reference herein. In one aspect, a binding polypeptide may comprise an Fc variant comprising an amino acid substitution at EU position 268 (e.g., H268D or H268E). In another aspect, a binding polypeptide may comprise an amino acid substitution at EU position 239 (e.g., S239D or S239E) and/or EU position 332 (e.g., I332D or I332Q).

In certain aspects, the Fc region of the anti-CTLA-4 binding moieties of the multispecific binding proteins of the current disclosure can comprise one or more mutations to decrease or eliminate the effector functions (see, for example, Zhou et al. (2020) mAbs 12(1): 1814583, which is incorporated by reference herein in its entirety). In some aspects, at least one Fc region comprises one or more substitutions at amino acid positions 114, 298, 299, and/or 300 according to the Kabat numbering (e.g., the NNAS mutant—i.e. containing the S298N/T299A/Y300S mutations, or for e.g., the A114N glycosylation mutant, A114N).

In certain aspects, the Fc region of the anti-CTLA-4 binding moieties of the multispecific binding proteins of the current disclosure comprise one or more mutations to modulate half-life (See e.g., Dall'Acqua et al. (2006) J Biol Chem 281:23514-24, Zalevsky et al. (2010) Nat Biotechnol 28:157-9, Hinton et al. (2004) J Biol Chem 279:6213-6, Hinton et al. (2006) J Immunol 176:346-56, Shields et al. (2001) J Biol Chem 276:6591-604, Petkova et al. (2006) Int Immunol 18:1759-69, Datta-Mannan et al. (2007) Drug Metab Dispos 35:86-94, Vaccaro et al. (2005) Nat Biotechnol 23:1283-8, Yeung et al. (2010) Cancer Res 70:3269-77 and Kim et al. (1999) Eur J Immunol 29:2819-25. (e.g., T250Q, M252Y, I253A, S254T, T256E, P257I, T307A, D376V, E380A, M428L, H433K, N434S, N434A, N434H, N434F, H435A and/or H435R).

In certain aspects, the multispecific binding proteins of the current disclosure can have a modified Fc domain. In certain aspects, the multispecific binding proteins of the current disclosure can have a tyrosine (Y) at amino acid position 252, according to EU numbering. In certain aspects, a conjugate can have an aspartic acid (D) or a glutamic acid (E) at amino acid position 256, according to EU numbering. In certain aspects, the multispecific binding proteins of the current disclosure can have a tryptophan (W) or a glutamine (Q) at amino acid position 307, according to EU numbering. In certain aspects, a conjugate can have a phenylalanine (F) or a tyrosine (Y) at amino acid position 434; according to EU numbering.

In certain aspects, the multispecific binding proteins of the current disclosure can have a modified Fc domain comprising any combination of the following four amino acid residues: a tyrosine (Y) at amino acid position 252, an aspartic acid (D) or a glutamic acid (E) at amino acid position 256, a tryptophan (W) or a glutamine (Q) at amino acid position 307, and a phenylalanine (F) or a tyrosine (Y) at amino acid position 434; according to EU numbering.

In certain aspects, the multispecific binding proteins of the current disclosure can comprise a modified Fc domain having combination of amino acid residues selected from the group consisting of: a) a tyrosine (Y) at amino acid position 252, an aspartic acid (D) at amino acid position 256, a glutamine (Q) at amino acid position 307, and a tyrosine (Y) at amino acid position 434; b) a tyrosine (Y) at amino acid position 252, a glutamic acid (E) at amino acid position 256, a tryptophan (W) at amino acid position 307, and a tyrosine (Y) at amino acid position 434; c) a tyrosine (Y) at amino acid position 252, a glutamic acid (E) at amino acid position 256, a glutamine (Q) at amino acid position 307, and a tyrosine (Y) at amino acid position 434; d) a tyrosine (Y) at amino acid position 252, an aspartic acid (D) at amino acid position 256, a glutamine (Q) at amino acid position 307, and a phenylalanine (F) at amino acid position 434; e) a tyrosine (Y) at amino acid position 252, an aspartic acid (D) at amino acid position 256, a tryptophan (W) at amino acid position 307, and a tyrosine (Y) at amino acid position 434; and f) a tyrosine (Y) at amino acid position 252, an aspartic acid (D) at amino acid position 256, a tryptophan (W) at amino acid position 307, and a phenylalanine (F) at amino acid position 434; according to EU numbering.

In certain aspects, the multispecific binding proteins of the current disclosure can comprise a modified Fc domain comprising a quadruple amino acid substitution selected from the group consisting of: M252Y/T256D/T307Q/N434Y, M252Y/T256E/T307W/N434Y, M252Y/T256E/T307Q/N434Y, M252Y/T256D/T307Q/N434F, M252Y/T256D/T307W/N434Y, and M252Y/T256D/T307W/N434F; according to EU numbering.

In some aspects, the multispecific binding protein comprises one or more mutations or glycan modifications to modulate the Fc mediated effector function. In some aspects, the multispecific binding protein comprises one or more mutations to modulate half-life.

### VI. Species Selectivity and Species Cross-Reactivity

In certain aspects, the multispecific binding proteins of the disclosure employ CTLA binding moieties which bind to human CTLA-4 but not to CTLA-4 from other species. Alternatively, the multispecific binding protein employ a CTLA-4 binding moiety which binds to human CTLA-4 and to CTLA-4 from one or more non-human species. For example, the multispecific binding protein can bind to human CTLA-4 and can bind or not bind, as the case can be, to one or more of mouse, rat, guinea pig, hamster, gerbil, pig, cat, dog, rabbit, goat, sheep, cow, horse, camel, cynomolgus, marmoset, rhesus or chimpanzee CTLA-4. In certain aspects, the multispecific binding protein can bind to human CTLA-4, but do not bind to rat and mouse CTLA-4. In other aspects, the multispecific binding protein bind to human CTLA-4, and to rat and mouse CTLA-4, with similar binding affinities.

### VII. Target Protein Binding Moieties

The multispecific binding proteins of the disclosure further comprise a binding moiety which binds a target protein of interest. As the skilled artisan will appreciate, when paired with the first CTLA-4 binding moiety to form a multispecific binding protein of the disclosure, the second binding moiety of the multispecific binding protein can facilitate the internalization and lysosomal degradation of the target protein of interest to which it binds. By specifically binding to a target protein, the multispecific binding protein enables its internalization by the T-cell. The internalized target protein is then transported to the lysosomal compartment, where it undergoes degradation. This mechanism provides a means to target and degrade various target proteins or target proteins.

As used herein a “target protein” is a protein having a deleterious function for which degradation would be therapeutically advantageous. In certain aspects, the target protein is a pathogenic protein or a peptide which causes a disease or symptom of disease. In certain aspects, the target protein is a membrane target protein. In certain aspects, a target protein is a membrane-bound target protein co-expressed on a T-cell along with CTLA-4. In other aspects, the target protein is expressed on the surface of a non-T cell (e.g., an antigen presenting cell). In other aspects, the target protein is a soluble protein. Example target proteins or peptides include proteins or peptides secreted by tumors, inflammatory protein or peptides; signaling molecules including cytokines, interleukins, interferons, tumor necrosis factors (TNF), growth factors, hormones, neurotransmitters, lipid mediators, activating factors, extracellular matrix (ECM) proteins, Wnt proteins, members of the Transforming Growth Factor-beta (TGF-β) family, Notch ligands; and the like.

In some aspects, the target protein is selected from the group consisting of an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

In certain aspects, the target protein is an antigen (e.g., an autoantigen). Antigens are molecules that can elicit an immune response. In certain aspects the antigen is an autoantigen or self-antigen produced in the cell of a subject. For example, an antigen could be a surface marker expressed on specific cell types, allowing a multispecific binding protein of the disclosure to selectively target and modulate those cells. By engaging the T cells via a CTLA-4 binding portion, antigen-targeting multispecific binding proteins of the disclosure can enhance immune responses, facilitate cell-mediated cytotoxicity, or regulate immune cell functions in immunotherapy.

In other aspects, the target protein is an antibody (e.g., an autoantibody) or fragment thereof. An autoantibody is an antibody that specifically binds to one or more antigens made or formed by a subject's own body. Autoantibodies mistakenly recognize and target self-antigens, leading to autoimmune diseases. By binding autoantibodies as the second binding moiety, the multispecific binding protein, or a binding fragment or derivative thereof can specifically bind to the self-antigens associated with autoimmune disorders. This approach offers the potential for targeted therapy by redirecting the immune response towards the autoreactive cells or molecules involved in the autoimmune process. In certain aspects, an autoantibody of the present disclosure is IgM Rheumatoid Factor (IgM-RF).

By targeting specific proteins, the multispecific binding proteins of the disclosure can, for example, interfere with protein-protein interactions, disrupt signaling pathways, or block protein-mediated cellular functions. In certain aspects, the target protein is membrane protein. Membrane proteins are a class of proteins located within or associated with cellular membranes, playing, for example, roles in cell signaling, transport of molecules across membranes, and maintaining the structural integrity of the cell. In certain aspects, the target protein is a soluble protein. Soluble proteins are a class of proteins that readily dissolve in aqueous environments, maintaining stability and performing diverse functions within the cellular context.

In certain aspects, the target protein is an immune checkpoint protein. In certain aspects, the target protein is associated with a disease or disorder where aberrant protein signaling is involved, such as certain cancers or metabolic disorders. The multispecific binding protein of the disclosure may be designed to target proteins with high affinity and selectivity, enabling precise modulation of the aberrant signaling pathway.

In certain aspects, a target protein is a pathogenic protein. Pathogenic proteins are those that are associated with disease development or progression. By facilitating degradation of target pathogenic protein, the multispecific binding protein of the disclosure, can neutralize their activity, inhibit their binding to receptors or other molecules, or facilitate their clearance from the body. This approach is relevant in the field of, e.g., infectious diseases, or chronic infectious diseases such as such as Herpes viral infection (HSV, CMV, EBV), HIV-1, and HBV infections. In some aspects, the multispecific binding proteins may be used to treating chronic viral infection. In certain aspects, the multispecific binding proteins of the current disclosure can be designed to target viral or bacterial proteins involved in pathogenesis. By blocking or neutralizing pathogenic proteins, the multispecific binding protein, can help control the spread of the disease and limit its impact on the host. In certain aspects, the multispecific binding protein can comprise the variable domains of, be co-administered with, or fused to an agent targeting an infectious disease target of interest. In certain aspects, the agent can be Palivizumab (e.g., to target the fusion (F) glycoprotein).

In certain aspects, a target protein can be a target protein secreted by tumors. Tumor cells can release proteins that contribute to tumor growth such as growth factors, angiogenesis, immune evasion, or metastasis. The multispecific binding proteins that target these proteins can interfere with their function, inhibit tumor-promoting activities, or enhance anti-tumor immune responses. This approach offers the potential for targeted therapy against cancer by specifically neutralizing or modulating tumor-secreted proteins that play critical roles in tumorigenesis and progression. In certain aspects, a target protein secreted by tumors of the present disclosure is Vascular endothelial growth factor A (VEGFA). In certain aspects, the multispecific binding protein can comprise the variable domains of, be co-administered with or fused to an agent targeting a particular tumor. An exemplary agent can include pegaptanib, bevacizumab, ranibizumab, brolucizumab, aflibercept (e.g., to target VEGFA). In some aspects, the target protein is a tumor secreted protein.

In certain aspects, a target protein can be an inflammatory protein. Inflammatory proteins are involved in the immune response and can contribute to chronic inflammation, autoimmune disorders, or tissue damage. The multispecific binding protein can be designed to target inflammatory proteins and help regulate the inflammatory cascade, suppress excessive immune responses, or modulate immune cell functions. By selectively binding and neutralizing inflammatory proteins, the multispecific binding proteins of the disclosure have the potential to dampen inflammation and restore immune balance in various inflammatory conditions. Examples of inflammatory conditions include rheumatoid arthritis, dermatitis, and systemic lupus erythematosis (SLE). In certain aspects, the multispecific binding protein can comprise the variable domains of, be co-administered with, or fused to an agent targeting a proinflammatory protein of interest. Example agents can include eculizumab or ravulizumab (e.g., to target complement component C5).

Interleukins (ILs) are a specific group of signaling molecules involved in immune responses and inflammation. The multispecific binding protein of the disclosure can be engineered to target specific ILs or their receptors, thereby modulating their activity and downstream signaling. This approach can be applied to various immune-related disorders, such as autoimmune diseases, allergies, or inflammatory conditions. By interfering with IL signaling, the multispecific binding proteins can regulate immune cell activation, cytokine production, or immune cell trafficking, providing a potential avenue for therapeutic intervention. In certain aspects, the multispecific binding proteins can comprise the variable domain of, be co-administered with or fused to an agent targeting an IL of interest. An exemplary agent can include Siltuximab (e.g., to target IL6), Mepolizumab or Reslizumab (e.g., to target IL5), Secukinumab or Ixekizumab (e.g. to target IL17A), Guselkumab, Tildrakizumab, or Risankizumab (e.g. to target the p19 subunit of IL23), Rilonacept (e.g. to target IL1A and IL1B) Canakinumab (e.g. to target IL1B), Ustekinumab (e.g. to target the p40 subunit of IL12 and IL23).

In certain aspects, a target protein can be a Wnt protein. Wnt proteins are a family of secreted signaling molecules that regulate cell proliferation, differentiation, and tissue development. Dysregulation of Wnt signaling is implicated in numerous diseases, including cancer, developmental disorders, and degenerative diseases. The multispecific binding protein targeting Wnt proteins can modulate their activity, block aberrant signaling pathways, or interfere with Wnt protein interactions. This approach offers potential therapeutic strategies for diseases driven by aberrant Wnt signaling. In certain aspects, the multispecific binding can comprise the variable domains, of, be co-administered with or fused to an agent targeting an Wnt protein of interest. Example agents can include Vantictumab (e.g., to target FZD1/2/5/7/8).

In certain aspects, a target protein can be a cytokine. In certain aspects, the cytokine can be member of the Transforming Growth Factor-beta (TGF-β). Members of the TGF-β family are a group of multifunctional cytokines involved in various cellular processes, including cell growth, differentiation, immune regulation, and tissue repair. Dysregulation of TGF-β signaling is associated with fibrosis, cancer progression, immune disorders, and other diseases. The multispecific binding protein designed to target TGF-β family members can modulate their signaling pathways, inhibit their effects on immune cells or stromal cells, or interfere with TGF-β ligand-receptor interactions. By modulating TGF-β signaling, the multispecific binding proteins hold potential therapeutic value for a range of diseases associated with TGF-β dysregulation. In certain aspects, a TGF-β cytokine of the present disclosure is TGF-β1. In other aspects, the cytokine can be a member of the insulin-like growth factors (IGF). Examples of IGF include IGF-1 and IGF-2. Other example cytokines include IgE and IgA. In certain aspects, the multispecific binding protein can comprise the variable domains of, be co-administered with or fused to an agent targeting a cytokine of interest. An example agent can include omalizumab (e.g., to target IgE).

In certain aspects, a target protein can be a Notch ligand. Notch ligands are cell surface proteins involved in cellular communication and tissue development. Dysregulation of Notch signaling is implicated in cancer, cardiovascular diseases, and neurodegenerative disorders. The multispecific binding protein of the disclosure can disrupt Notch signaling pathways, block ligand-receptor interactions, or modulate downstream gene expression. This approach offers potential therapeutic strategies for diseases driven by aberrant Notch signaling, with the goal of restoring normal cellular processes and tissue homeostasis.

In certain aspects of this disclosure, the target protein is expressed on the T-cell membrane (e.g., on the same T-cell that is expressing the CTLA-4). In other aspects, the target protein is expressed on activated T-cells and/or T regulatory (Treg) cells. In some aspects, the target protein comprises a membrane associated protein, including immune checkpoint proteins and receptors expressed on T-cell surface. In some aspects, the target protein is an immune checkpoint protein and the second binding moiety comprises an agonist or an antagonist immune checkpoint modulator (e.g., an agonist or an antagonist immune checkpoint inhibitor). In some aspects, the second binding moiety comprises an agonist or an antagonistic antibody or antigen-binding fragment thereof against a receptor involved in immune modulation. In some aspects, the second binding moiety comprises an agonist or an antagonistic ISV against a receptor involved in immune modulation.

In certain aspects, the target protein is associated with a disease selected from the group consisting of a cancer, an autoimmune disease, an inflammatory disorder, an infectious disease, and a neurodegenerative disorder. In certain aspects, the target protein is associated with the cancer. In certain aspects, the target protein is associated with the autoimmune disease. In certain aspects, target protein is associated with the inflammatory disorder.

### VIII. pH Sensitive Target Protein Binding

In certain aspects, the multispecific binding proteins of the disclosure comprise a target binding moiety which exhibits pH-dependent binding to target protein. In certain aspects, the pH-sensitive binding moiety facilitates dissociation of target protein from the multispecific binding protein within the lysosomal compartment, allowing target protein to be degraded in the lysosomal compartment and/or allowing the multispecific binding protein to recycle back to the cell surface where it can bind additional target protein for degradation. For example, the target protein binding moiety may comprise a Fab domain comprising one or more mutations which enhance or diminish binding to target protein under different pH conditions e.g., at acidic pH as compared to neutral pH. For example, the target protein binding portion of the multispecific antibody can comprise a mutation in the CH1, CL, V_{H}, or the V_{L} region of the Fab domain, wherein the mutation(s) decreases the affinity of the Fab domain to its antigen in an acidic environment (e.g., in tumor microenvironment where pH is about 7.2, 7.0, 6.8, 6.5, 6.3 or lower. Such mutations can result in an increase in serum half-life of the multispecific binding protein when administered to an animal.

In certain aspects, the sensitivity of target protein binding at acidic pH may be increased, whereby the anti-target protein binding moiety demonstrates reduced binding to target protein at lower pH. In one aspect, binding to target protein is reduced at a pH that reflects the endosomal compartment. In another aspect, binding to target protein is reduced at pH 5.5 relative to binding at neutral pH (pH 7.0). Such reduced binding at pH 5.5 may be 50% or more of the target protein binding observed at neutral pH. A change, such as a reduction or increase, in an anti-target protein binding activity described herein, such as binding, may be in comparison to a reference or wild-type antibody. The change may also be relative between two different pH levels of a particular antibody composition described herein. pH sensitive anti-target protein antibodies may be identified by testing the interaction between plate coated target protein and soluble target protein antibodies over a pH range of 4.5 to 7.0, and selecting antibodies with increased pH sensitivity such that reduced binding is observed at acidic pH. Examples of an anti-target protein antibody with reduced binding to target protein at acidic pH comprises replacing tyrosine with histidine within or near one or more CDR1-3 regions of at least one of a light chain and heavy chain variable region of the antibody. See WO2020214748A1, incorporated by reference in its entirety.

In certain aspects, the second binding moiety of the multispecific binding protein that binds to the target protein exhibits pH-dependent binding to the target protein. In some aspects, the multispecific binding protein exhibits reduced binding at acidic pH.

### IX. Methods of Degradation of a Target Molecule

The multispecific binding proteins described herein can be used for CTLA-4 mediated lysosomal degradation of target proteins. In certain aspects, the methods involve the use of a multispecific binding protein, that comprises a first binding moiety specific for membrane-bound CTLA-4 on T-cells and a second binding moiety specific for the target molecule. Binding of the multispecific binding protein to membrane-bound CTLA-4 triggers the internalization of the complex into T-cells through endocytosis, initiating a series of intracellular events.

Upon internalization, the CTLA-4 and target protein complex are trafficked to early endosomes, where sorting and trafficking processes occur. In the early endosomes, the target protein becomes separated from the multispecific binding protein, or a binding fragment or derivative thereof, allowing the target protein to enter the endosomal lumen while CTLA-4 is recycled back to the cell surface. The target protein is now contained within the endosome.

The next stage involves the maturation of the endosome into late endosomes and subsequently into endolysosomes. Within these compartments, the target protein encounters an increasingly acidic environment, facilitated by the action of proton pumps. The acidic pH triggers the activation of lysosomal enzymes, such as proteases and nucleases, leading to the breakdown of the target protein into smaller peptides and ultimately to its complete degradation.

After degradation, the resulting peptides, along with any residual fragments of the multispecific binding protein, or a binding fragment or derivative thereof are subject to further processing within the endolysosomes. Some peptides can be presented on major histocompatibility complex (MHC) molecules for antigen presentation, contributing to immune surveillance and response. Meanwhile, CTLA-4, following its recycling pathway, is transported back to the cell surface, where it can engage with additional multispecific binding proteins, or a binding fragment or derivative thereof, and target proteins to initiate further rounds of internalization and degradation.

By exploiting the CTLA-4 shuttling mechanism, the methods enable the efficient and selective degradation of various target proteins, including pathogenic proteins, proteins secreted by tumors, autoantibodies, inflammatory proteins, interleukins, and signaling molecules. The disclosed methods can be used for the development of targeted therapies with precise control over the degradation of specific molecules for the treatment of diseases such as autoimmune disorders, cancer, and inflammatory conditions.

By facilitating the degradation of target proteins, the multispecific binding proteins of the disclosure are useful, inter alia, for the treatment, prevention and/or amelioration of any disease or disorder associated with or mediated by target protein expression, signaling, or activity, or treatable by CTLA-4-mediated degradation of the target protein within the lysosome. For example, the present disclosure provides methods for treating autoimmune disease, cancer (tumor growth inhibition), chronic viral infections, and other disease by administering the multispecific binding proteins described herein to a patient in need of such treatment. The multispecific binding proteins of the present disclosure are useful for the treatment, prevention, and/or amelioration of disease or disorder or condition such as autoimmune disease, a viral infection, or cancer and/or for ameliorating at least one symptom associated with such disease, disorder or condition. In the context of the methods of treatment described herein, the multispecific binding proteins can be administered as a monotherapy (i.e., as the only therapeutic agent) or in combination with one or more additional therapeutic agents (examples of which are described elsewhere herein).

In one aspect, this disclosure provides a method for degrading a target protein, comprising: contacting a T-cell with a multispecific binding protein, wherein the multispecific binding protein comprises: a) a first cell surface binding moiety that specifically binds to membrane-bound CTLA-4 on the surface of the T-cell; and b) a second binding moiety that is operatively linked to the first cell surface binding moiety and that specifically binds to the target protein, wherein binding of the multispecific binding protein to the membrane-bound CTLA-4 on the surface of the T-cell facilitates the internalization of the target protein by the T-cell.

In certain aspects, after internalization the target protein is degraded in a lysosome. In certain aspects, the multispecific binding protein exhibits increased degradation of the target protein compared to a reference binding polypeptide.

In some aspects, the multispecific binding protein, or a binding fragment or derivative thereof described herein are useful for treating subjects suffering from cancer or autoimmune and/or inflammatory disorders.

In certain aspects, the multispecific binding proteins of the disclosure are useful to treat subjects suffering from a chronic viral infection. In some aspects, the multispecific binding proteins are useful in decreasing target protein titers in the host via T-cell membrane bound CTLA-4. In some aspects, a multispecific binding proteins can be administered at a therapeutic dose to a patient suffering from an autoimmune disease, cancer or a viral infection.

### Reference Binding Polypeptide

In certain aspects, a multispecific binding protein of the disclosure exhibits increased internalization and/or degradation of the target protein compared to a reference binding polypeptide. In certain aspects, the reference binding polypeptide does not comprise the first cell surface binding moiety that specifically binds to cell surface CTLA-4 but is otherwise identical to the multispecific binding protein. In certain aspects, a multispecific binding protein exhibits increased degradation of the target protein by at least 2, 3, 4, 5, 10, 20, 30, 40, 50, 60, 70, 80, 90, or 100 percent compared to the reference binding polypeptide. In certain aspects, a multispecific binding protein degrades the target protein in at least 2, 3, 4, 5, 10, 24, 48, or 72 hours faster compared to the reference binding polypeptide.

### Specificity

The term “specificity” refers to the ability to specifically bind (e.g., immunoreact with) a given target antigen (e.g., cell surface CTLA-4). A subject multispecific binding protein contains two or more binding sites (e.g., 2, 3, 4, 5, or more) which specifically bind the same or different binding sites. In certain aspects, a subject multispecific binding protein is specific for two different (e.g., non-overlapping) target binding sites.

In certain aspects, the multispecific binding proteins of the disclosure employ cell surface CTLA-4 binding moieties which bind to human CTLA-4 but not to CTLA-4 from other species. Alternatively, the multispecific binding protein employ a CTLA-4 binding moiety which binds to human CTLA-4 and to CTLA-4 from one or more non-human species. For example, the multispecific binding protein can bind to human CTLA-4 and can bind or not bind, as the case can be, to one or more of mouse, rat, guinea pig, hamster, gerbil, pig, cat, dog, rabbit, goat, sheep, cow, horse, camel, cynomolgus, marmoset, rhesus or chimpanzee CTLA-4. In certain aspects, the multispecific binding protein can bind to human CTLA-4, but do not bind to rat and mouse CTLA-4. In other aspects, the multispecific binding protein bind to human CTLA-4, and to rat and mouse CTLA-4, with similar binding affinities.

### About or Approximately

The term “about” or “approximately” means within about 20%, such as within about 10%, within about 5%, or within about 1% or less of a given value or range.

### Administration

As used herein, “administer” or “administration” refers to the act of injecting or otherwise physically delivering a substance as it exists outside the body (e.g., a multispecific binding protein provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and can be continued chronically to defer or reduce the appearance or magnitude of disease-associated symptoms.

In certain aspects, the multispecific binding protein described herein is administered by intravenous, subcutaneous, intramuscular, or intradermal injection.

### Composition

As used herein, the term “composition” is intended to encompass a product containing the specified ingredients (e.g., a multispecific binding protein composition provided herein) in, optionally, the specified amounts, as well as any product which results, directly or indirectly, from combination of the specified ingredients in, optionally, the specified amounts.

In some aspects, a pharmaceutical composition comprises the multispecific binding protein described herein and a pharmaceutically acceptable carrier.

### Effective Amount

“Effective amount” means the amount of active pharmaceutical agent (e.g., a multispecific binding protein of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

In certain aspects, the disclosure provides a method of treating a subject with a disease associated with the target protein or soluble target protein, comprising administering to the subject a therapeutically effective amount of the multispecific binding protein described herein or a pharmaceutical composition comprising the multispecific binding protein described herein. In certain aspects, the method comprises binding of the multispecific binding protein to the membrane-bound CTLA-4 on the surface of the T-cell which facilitates the internalization and the trafficking of the target protein by the T-cell to a lysosome within the T-cell, enabling degradation of the target protein within the lysosome, thereby treating the disease in the subject. In some aspects, the disease is selected from the group consisting of a cancer, an autoimmune disease, an inflammatory disorder, an infectious disease, and a neurodegenerative disorder.

### Subject or Patient

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain aspects, the term “subject,” as used herein, refers to a vertebrate, such as a mammal. Mammals include, without limitation, humans, non-human primates, wild animals, feral animals, farm animals, sport animals, and pets.

### Therapy and Pharmaceutical Compositions

As used herein, the term “therapy” refers to any protocol, method and/or agent that can be used in the prevention, management, treatment and/or amelioration of a disease or a symptom related thereto. In some aspects, the term “therapy” refers to any protocol, method and/or agent that can be used in the modulation or depletion of a target protein from the circulation of a subject. In some aspects, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other aspects, the terms “therapies” and “therapy” refer to a biological therapy, supportive therapy, and/or other therapies useful in the modulation of an immune response to an inflammatory or autoimmune diseases in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as the administration of a multispecific binding protein provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

### Polynucleotides

In one aspect, polynucleotides (i.e., nucleic acid molecules) encoding a multispecific binding protein described herein or variants thereof are provided. A polynucleotide variant as used herein is about 50, 75, 80, 85, 90, 93, 95, 98, 99% or more identical to a polynucleotide that encodes a multispecific binding protein described herein.

Methods of making a multispecific binding protein comprising expressing these polynucleotides are also provided. Polynucleotides encoding a multispecific binding protein or variants thereof disclosed herein are typically inserted in an expression vector for introduction into host cells that can be used to produce the desired quantity of the claimed multispecific binding protein. Accordingly, in certain aspects, the disclosure provides expression vectors comprising polynucleotides disclosed herein and host cells comprising these vectors and polynucleotides.

In certain aspects, nucleic acid molecules encode an amino acid sequence for a) a first cell surface binding moiety that specifically binds to CTLA-4 on the surface of a CTLA-4 positive cell (e.g., a CTLA-4 expressed on the surface of a T cell); and b) a second binding moiety that is operatively linked to the first cell surface binding moiety and that specifically binds to a target protein.

In certain aspects, an isolated nucleic acid molecule encodes the multispecific binding protein disclosed herein.

### Expression Vector and Host Cell

The term “vector” or “expression vector” is used herein for the purposes of the specification and claims, to mean vectors used in accordance with the present disclosure as a vehicle for introducing into and expressing the polynucleotide sequence encoding a multispecific binding protein polypeptide in a cell. Such vectors include, for example, plasmids, phages, viruses and retroviruses. In general, vectors compatible with the instant disclosure will comprise a selection marker, appropriate restriction sites to facilitate cloning of the desired gene and the ability to enter and/or replicate in eukaryotic or prokaryotic cells.

In vitro production allows scale-up to give large amounts of the desired polypeptides. Techniques for mammalian cell cultivation under tissue culture conditions include, for example, homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more genes encoding a multispecific binding protein can also be expressed non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella, Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides can be isolated, purified and then assembled into functional molecules.

In addition to prokaryotes, eukaryotic cells can also be used. Saccharomyces cerevisiae, or common baker's yeast, is the most commonly used although a number of other strains are commonly available.

In some aspects, a vector comprises nucleic acid molecules encoding an amino acid sequence for a) a first cell surface binding moiety that specifically binds to CTLA-4 on the surface of a CTLA-4 positive cell; and b) a second binding moiety that is operatively linked to the first cell surface binding moiety and that specifically binds to a target protein.

In some aspects, at least two vectors comprise nucleic acid molecules encoding an amino acid sequence for a) a first cell surface binding moiety that specifically binds to CTLA-4 on the surface of a CTLA-4 positive cell; and b) a second binding moiety that is operatively linked to the first cell surface binding moiety and that specifically binds to a target protein.

In some aspects, two vectors comprise a multispecific binding protein of the disclosure. In some aspects, the first vector comprises a nucleic acid molecule encoding an amino acid sequence for a) a first cell surface binding moiety that specifically binds to CTLA-4 on the surface of a CTLA-4 positive cell. In some aspects, the second vector comprises a second binding moiety that is operatively linked to the first cell surface binding moiety and that specifically binds to a target protein. In some aspects, two vectors comprise a multispecific binding protein of the disclosure.

In some aspects, a cell comprises a vector comprising a nucleic acid molecule encoding an amino acid sequence for a) a first cell surface binding moiety that specifically binds to CTLA-4 on the surface of a CTLA-4 positive cell; and b) a second binding moiety that is operatively linked to the first cell surface binding moiety and that specifically binds to a target protein. In some aspects, a cell comprises two vectors comprising nucleic acid molecules encoding an amino acid sequence for a) a first cell surface binding moiety that specifically binds to CTLA-4 on the surface of a CTLA-4 positive cell; and b) a second binding moiety that is operatively linked to the first cell surface binding moiety and that specifically binds to a target protein In some aspects, a cell comprises at least two vectors comprising nucleic acid molecules encoding an amino acid sequence for a) a first cell surface binding moiety that specifically binds to CTLA-4 on the surface of a CTLA-4 positive cell; and b) a second binding moiety that is operatively linked to the first cell surface binding moiety and that specifically binds to a target protein.

In some aspects, an expression vector comprises the nucleic acid molecule encoding the first cell surface binding moiety of the second binding moiety that binds to the target protein. In some aspects, a host cell comprises said expression vector.

It is to be understood that this disclosure is not limited to particular methods and experimental conditions described, as such methods and conditions can vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value can vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, exemplary methods and materials are now described. All publications mentioned herein are incorporated herein by reference to describe in their entirety.

### EXAMPLES

The following examples are put forth so as to provide those of ordinary skill in the art with a complete disclosure and description of how to make and use the methods and compositions featured in the disclosure and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Design of Anti-TNFα/CTLA-4 Bispecific Antibody Constructs

Bispecific antibody constructs that were specific to both a target protein (e.g., a TNFα) and cell surface expressing CTLA-4 were designed. The light and heavy chain amino acid sequences are shown below in Table 2.

TABLE 2
Anti-TNFα/CTLA-4 bispecific antibody light
and heavy chain amino acid sequences

| SEQ ID | |
|---|---|
| LC1 | DIQMTQSPSSLSASVGDRVTITCRASQGIRNYLAWYQQKPGKAPKLLIY |
| (a-TNFα) | AASTLQSGVPSRFSGSGSGTDFTLTISSLQPEDVATYYCQRYNRAPYTF |
| (SEQ ID | GQGTKVEIKRTVAAPSVFIFPPSDEQLKSGTASVVCLLNNFYPREAKVQ |
| NO: 1) | WKVDNALQSGNSQESVTEQDSKDSTYSLSSTLTLSKADYEKHKVYAC |
| | EVTHQGLSSPVTKSFNRGEC |
| HC1 | EVQLVESGGGLVQPGRSLRLSCAASGFTFDDYAMHWVRQAPGKGLE |
| (a-TNFα) | WVSAITWNSGHIDYADSVEGRFTISRDNAKNSLYLQMNSLRAEDTAV |
| (SEQ ID | YYCAKVSYLSTASSLDYWGQGTLVTVSSASTKGPSVFPLAPSSKSTSG |
| NO: 2) | GTAALGCLVKDYFPEPVTVSWNSGALTSGVHTFPAVLQSSGLYSLSSV |
| | VTVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKAL |
| | PAPIEKTISKAKGQPREPQVYTLPPSRDELTKNQVSLWCLVKGFYPSDI |
| | AVEWESNGQPENNYKTTPPVLDSDGSFFLYSKLTVDKSRWQQGNVFS |
| | CSVMHEALHNHYTQKSLSLSPG |
| LC2 | EIVLTQSPGTLSLSPGERATLSCRASQSVGSSYLAWYQQKPGQAPRLLI |
| (a-CTLA-4) | YGAFSRATGIPDRFSGSGSGTDFTLTISRLEPEDFAVYYCQQYGSSPWT |
| (SEQ ID | FGQGTKVEIKRTVAAPSVFIFPPSDEQLKSGTASVVCLLNNFYPREAKV |
| NO: 3) | QWKVDNALQSGNSQESVTEQDSKDSTYSLSSTLTLSKADYEKHKVYA |
| | CEVTHQGLSSPVTKSFNRGEC |
| HC2 | QVQLVESGGGVVQPGRSLRLSCAASGFTFSSYTMHWVRQAPGKGLEW |
| (a-CTLA-4) | VTFISYDGNNKYYADSVKGRFTISRDNSKNTLYLQMNSLRAEDTAIYY |
| (SEQ ID | CARTGWLGPFDYWGQGTLVTVSSASTKGPSVFPLAPSSKSTSGGTAAL |
| NO: 4) | GCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIE |
| | KTISKAKGQPREPQVYTLPPSRDELTKNQVSLSCAVKGFYPSDIAVEWE |
| | SNGQPENNYKTTPPVLDSDGSFFLVSKLTVDKSRWQQGNVFSCSVMH |
| | EALHNHYTQKSLSLSPG |
| HC2 | VIHVTKEVKEVATLSCGHNVSVEELAQTRIYWQKEKKMVLTMMSGD |
| (CD80-ECD | MNIWPEYKNRTIFDITNNLSIVILALRPSDEGTYECVVLKYEKDAFKRE |
| HC Fusion) | HLAEVTLSVKADFPTPSISDFEIPTSNIRRIICSTSGGFPEPHLSWLENGEE |
| (SEQ ID | LNAINTTVSQDPETELYAVSSKLDFNMTTNHSFMCLIKYGHLRVNQTF |
| NO: 5) | NWNTTKQEHFPDNEPKSCDKTHTCPPCPAPELLGGPSVFLFPPKPKDTL |
| | MISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTT |
| | PPVLDSDGSFFLASKLTVDKSRWQQGNVFSCSVMHEALHNHYTQKSL |
| | SLSPG |
| HC2 | APLKIQAYFNETADLPCQFANSQNQSLSELVVFWQDQENLVLNEVYLG |
| (CD86-ECD | KEKFDSVHSKYMGRTSFDSDSWTLRLHNLQIKDKGLYQCIIHHKKPTG |
| HC Fusion) | MIRIHQMNSELSVLANFSQPEIVPISNITENVYINLTCSSIHGYPEPKKMS |
| (SEQ ID | VLLRTKNSTIEYDGVMQKSQDNVTELYDVSISLSVSFPDVTSNMTIFCIL |
| NO: 6) | ETDKTRLLSSPFSIELEDPQPPPDHIPEPKSCDKTHTCPPCPAPELLGGPS |
| | VFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWE |
| | SNGQPENNYKTTPPVLDSDGSFFLASKLTVDKSRWQQGNVFSCSVMH |
| | EALHNHYTQKSLSLSPG |

### Example 2: Cellular Internalization and Degradation of TNFα Via an Anti-TNFα/CTLA-4 Bispecific Antibody

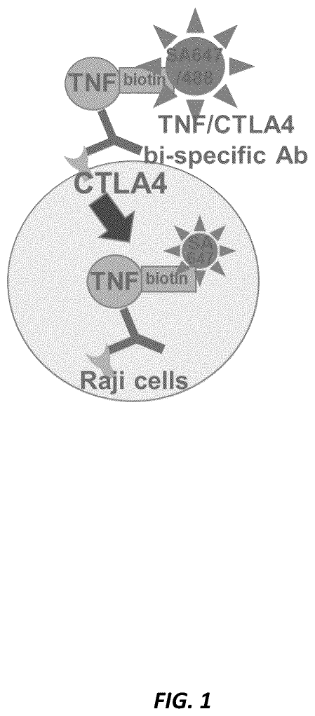

Example 2 tests the ability of the anti-TNFα/CTLA-4 bispecific antibody designed in Example 1 to facilitate cellular internalization and degradation of the target protein (e.g., TNFα) as schematized in FIG. 1.

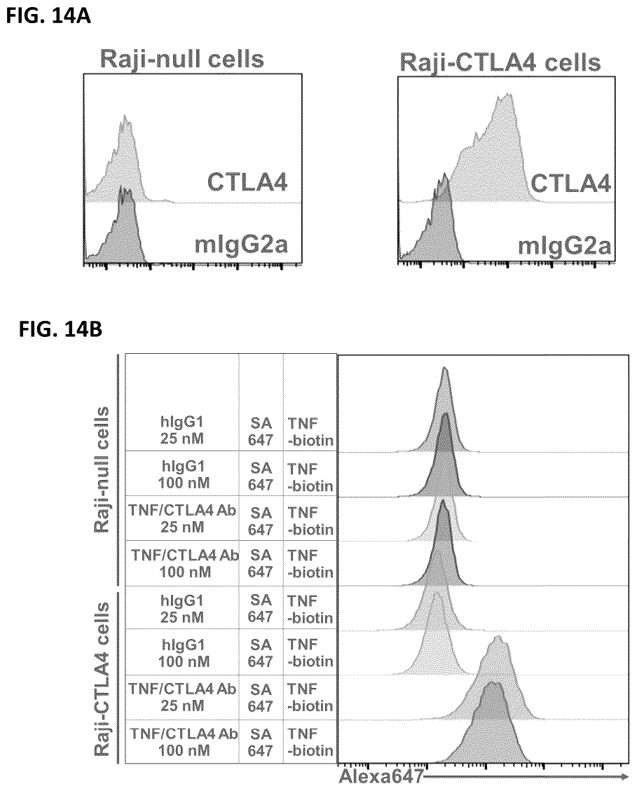

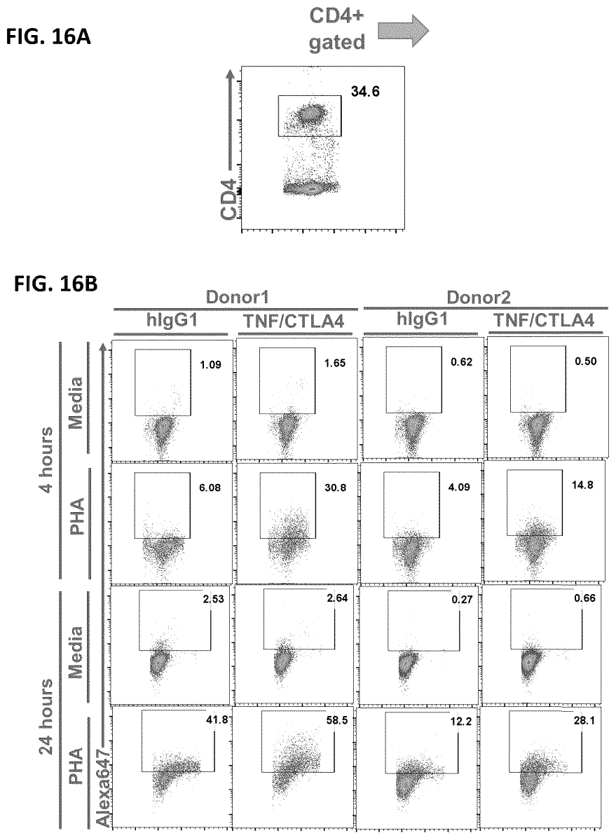

Most experiments in Example 2 were performed utilizing a wild-type Raji cell line (Raji-null cells) or a Raji cell line that was engineered to stably express human CTLA-4 on the cell surface membrane (Raji-CTLA-4 cells) (see CTLA-4 specific MFI shift in FIG. 14A). To confirm results were not dependent on the Raji cell line, experiments were also performed using activated PBMCs from two different human donors (“donor 1” and donor 2″; see FIGS. 16A and 16B).

### Methods

CTLA-4 Internalization with an Anti-CLTA-4 Antibody Analysis by Confocal Microscope

Raji cells or Raji cells that stably expressed human CTLA-4 were incubated with 75 UM of anti-CTLA-4 antibody (ipilimumab) labeled with Alexa Fluor 488 at 37° C. for 1 and 4 hours in cell culture media in the dark. After the incubation, the media were removed, and the cells were washed with PBS. Cells were then fixed with 4% paraformaldehyde (in 0.1 M phosphate buffer, at pH 7.4) at room temperature for 1 hour, permeabilized with 0.5% Triton X-100 in PBS, washed three times with PBS, stained with the nuclear dye, 4′,6-diamidino-2-phenylindole (DAPI) (Thermo Fisher Scientific, Waltham, MA), and mounted using AquaPoly/Mount.

To protect against photobleaching during the fixation and staining process, all incubations were carried out in the dark as described previously (Piepenhagen, Microsc Res Tech. 2010). The permeabilized cells were also incubated with fluorescence labeled anti-Early Endosome Antigen 1 (EEA1) or LAMP-1 antibodies, respectively.

All micrographs were acquired using a Zeiss LSM880 confocal microscope (Carl Zeiss, White Plains, NY) equipped with a 40× Plan-Apo water immersion objective. Alexa Fluor 488 was excited using the 488-nm line of an argon laser and detected using a 515-565-nm band pass filter. Other fluorescent labels were excited using different nm laser lines and were detected using different nm band-pass filters. One or two fields were randomly picked per sample and optical stacks were recorded. All images were acquired using identical parameters.

CTLA-4 Internalization with an Anti-TNFα/CTLA-4 Bispecific Antibody Analysis by Confocal Microscope

Raji-null cells or Raji-CTLA-4 cells that stably expressed human CTLA-4 (as shown in FIG. 14A) were incubated with the following bispecific antibody complex: 25 nM anti-TNFα/CTLA-4 bispecific antibody, 50 nM biotinylated TNFα, and 100 nM streptavidin conjugated to AlexaFlour 488 at 37° C. for a 1-, 2-, or 4-hour time course in cell culture media in the dark. After the incubation, the media was removed and the cells were washed with PBS.

Cells were then fixed with 4% paraformaldehyde (in 0.1 M phosphate buffer, at pH 7.4) at room temperature for 30 minutes and cytospun onto glass slides. Samples were then permeabilized with 0.5% Triton X-100 in PBS, washed three times with PBS, and stained and incubated with primary rabbit anti-Early Endosome Antigen 1 (EEA1, endosome marker) and mouse anti-LAMP-1 (lysosome marker) antibodies. After incubation with primary antibodies, samples were washed three times with PBS and stained and incubated with hoeschst (nucleus marker) as well as with fluorescent goat anti-rabbit and goat anti-mouse secondary antibodies. Following staining samples were washed and mounted using AquaPoly/Mount. To protect against photobleaching during the fixation and staining process, all incubations were carried out in the dark as described previously (Piepenhagen et al., Use of direct fluorescence labeling and confocal microscopy to determine the biodistribution of two protein therapeutics, Cerezyme and Ceredase. Microsc Res Tech. 2010 July; 73(7):694-703).

All micrographs were acquired using a Zeiss LSM880 confocal microscope (Carl Zeiss, White Plains, NY) equipped with a 40× Plan-Apo water immersion objective. Alexa Fluor 488 was excited using the 488-nm line of an argon laser and detected using a 515-565-nm band pass filter. Other fluorescent labels were excited using different nm laser lines and were detected using different nm band-pass filters. High magnification images were captured using the Zeiss Airyscan detector array to acquire each channel sequentially. One or two fields were randomly picked per sample and optical stacks were recorded. All images were acquired using identical parameters. Images were analyzed in Cellprofiler (Cimini lab, Broad Institute) using maximum intensity projections of the optical stacks. Data were plotted and statistics were calculated using RStudio (version 2022.12.0, Posit Software).

Live Cell Imaging with pH-Dependent Dye

Raji-null or Raji-CTLA-4 cells that stably express surface human CTLA-4 were plated at a concentration of 200,000 cells/well in 35 mm² glass-bottom microwell dishes that had been coated with poly-d-lysine at 1 mg/mL. Cells were allowed to rest overnight to attach to dishes. Recombinant biotinylated TNFα at a final concentration of 50 nM, streptavidin-pHrodo red at a final concentration of 100 nM, and anti-TNFα/CTLA-4 bispecific antibody at a final concentration of 25 nM were added to each dish immediately before imaging. Hoescht (nuclear dye) was added to media at a final dilution of 1:5000 and Lyso View633 dye (lysosome marker dye, Biotium, Inc.) was added to media of some samples at a final concentration of 1× from a 1000× stock solution. Dish was immediately taken for live cell imaging in a stage top incubator system set to 37° C. with 5% CO₂ and added humidity.

All micrographs were acquired using a Zeiss LSM780 confocal microscope (Carl Zeiss, White Plains, NY) equipped with a 20× Plan-Apo air immersion objective. Five fields of view were randomly chosen from each dish and optical stacks were acquired every 3 minutes for up to two hours. pHrodo red dye was excited using the 561 nm laser line and was detected using a bandpass filter. LysoView633 was excited using the 633 nm laser line and Hoescht was excited using the 405 nm laser line. They were detected using appropriate bandpass filters. Images were acquired using identical parameters. Live imaging data was analyzed in Cellprofiler (Cimini lab, Broad Institute) using maximum intensity projections of the optical stacks. Data were plotted and statistics were calculated using RStudio (version 2022.12.0, Posit Software).

### TNFα Internalization Assay by Flow Cytometry

Raji-null or Raji-CTLA-4 cells that stably expressed surface human CTLA-4 were plated at a concentration of 10⁵ cells/well in U-bottom 96 well plates. Recombinant biotinylated TNFα at a final concentration of 50 nM, streptavidin-Alexa647 at a final concentration of 100 nM, and anti-TNFα antibody or anti-TNFα/CTLA-4 bispecific antibody at a final concentration of 25 or 100 nM were added sequentially to the 96-well plate. After the cells were cultured at 37° C. for 4 hours, they were washed twice with cold phosphate buffered saline (PBS), then flow cytometry was conducted to assess Alexa647 fluorescence. The same method was used for anti-TNFα/CTLA-4 bi-specific antibody at a final concentration of 25 nM with 24-hour PHA-stimulated peripheral blood mononuclear cells (PBMC). Flowcytometry data were analyzed with FlowJo.

### TNFα Degradation Assay by Western Blotting

Raji-CTLA4 cells were plated at a concentration of 10⁵ cells/well in U-bottom 96 well plates. Recombinant TNFα at a final concentration of 50 nM, and anti-TNFα antibody or anti-TNFα/CTLA-4 bi-specific antibody at a final concentration of 25 nM were added sequentially to the 96-well plate. After the cells were cultured at 37° C. for 2 hours, they were washed twice with media and lysate was produced using radioimmunoprecipitation assay (RIPA) buffer. A few cells remained in culture for an additional 2, 9 or 22 hours in the presence of DMSO or 100 nM Bafilomycin A. At each time point, the cells were washed twice with cold PBS then RIPA buffer to produce a cell lysate. Western blotting was employed to detect TNFα or β-actin with the prepared lysate.

### Results

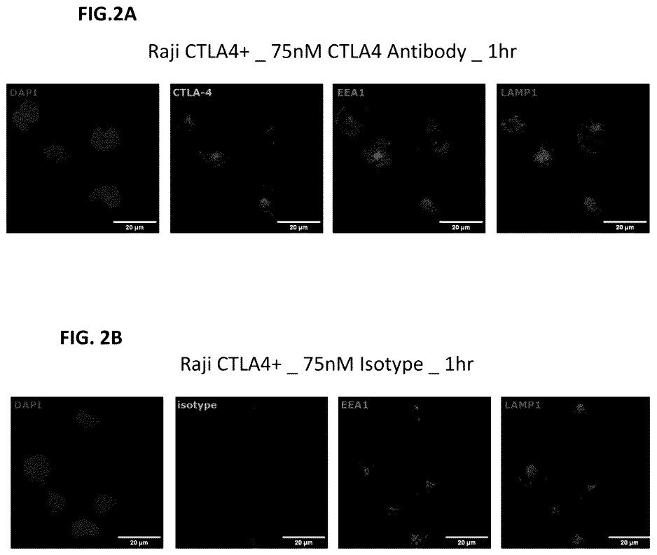

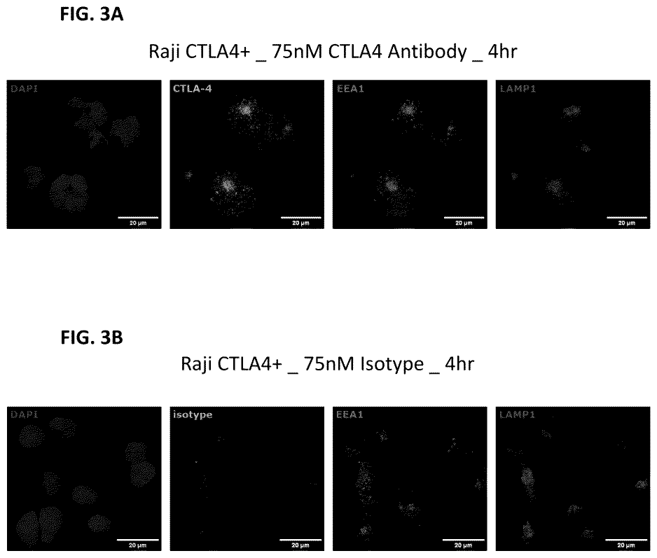

CTLA-4 was rapidly internalized after the CTLA-4 expressing cells were incubated with anti-CTLA-4 antibody but not with isotype control for one hour or for four hours as determined by confocal microscopy (FIGS. 2A, 2B, 3A, and 3B).

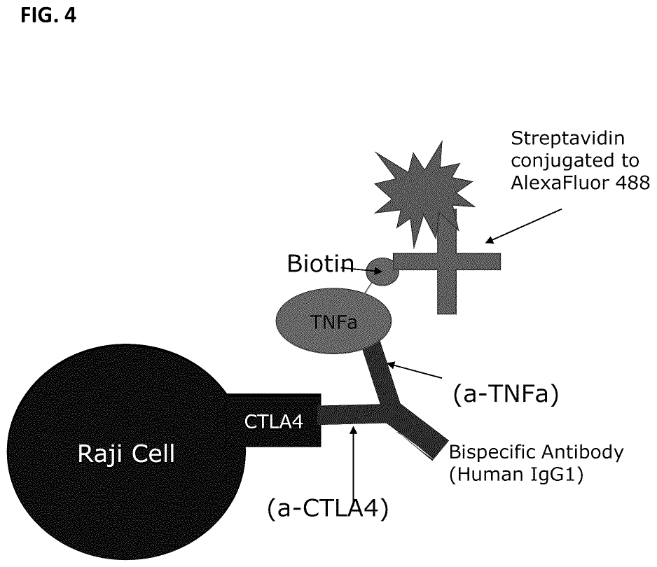

To assess via confocal microscopy the cellular internalization and lysosomal trafficking steps of the target protein, TNFα, after binding to an anti-TNFα/CTLA-4 bispecific antibody construct described in Example 1, Raji-null or Raji-CTLA-4 cells (FIG. 14A) were incubated with a bispecific antibody complex which comprised biotinylated human TNFα, streptavidin conjugated to AlexaFluor488 (streptavidin-AF488), and an anti-TNFα/CTLA-4 bispecific antibody as depicted in FIG. 4.

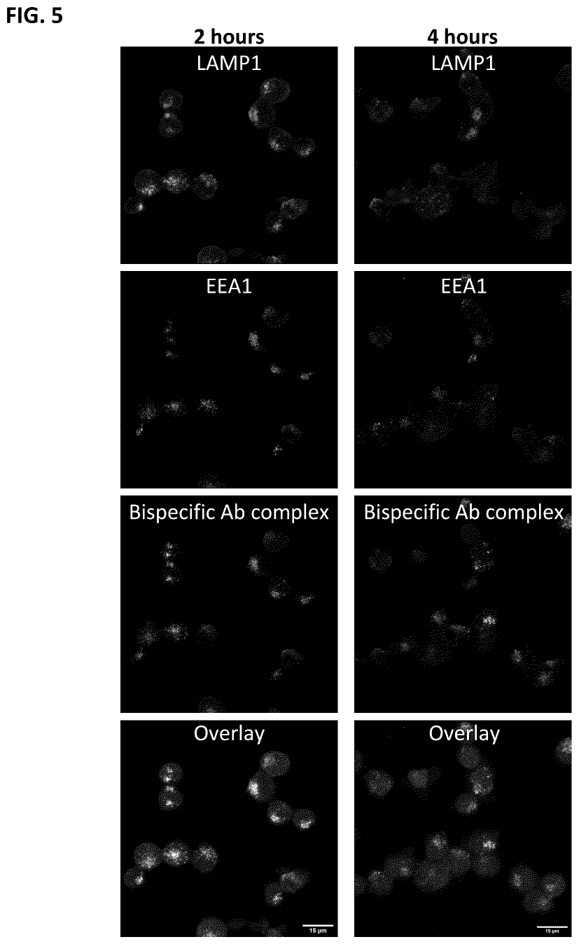

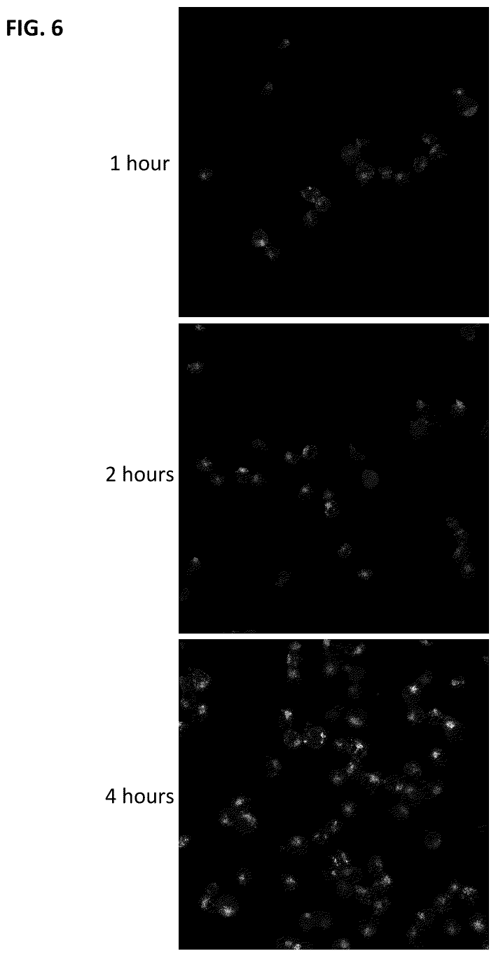

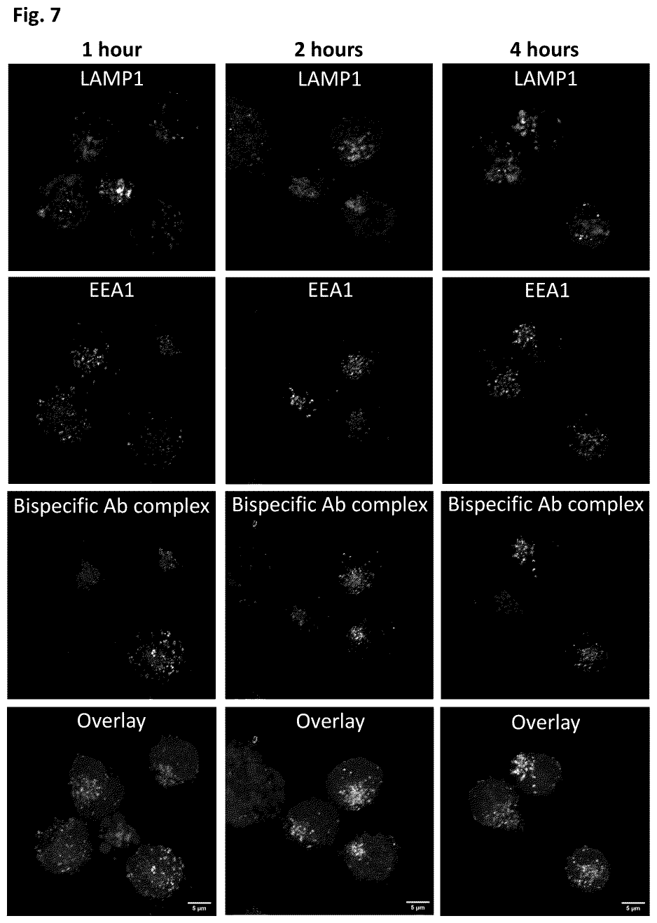

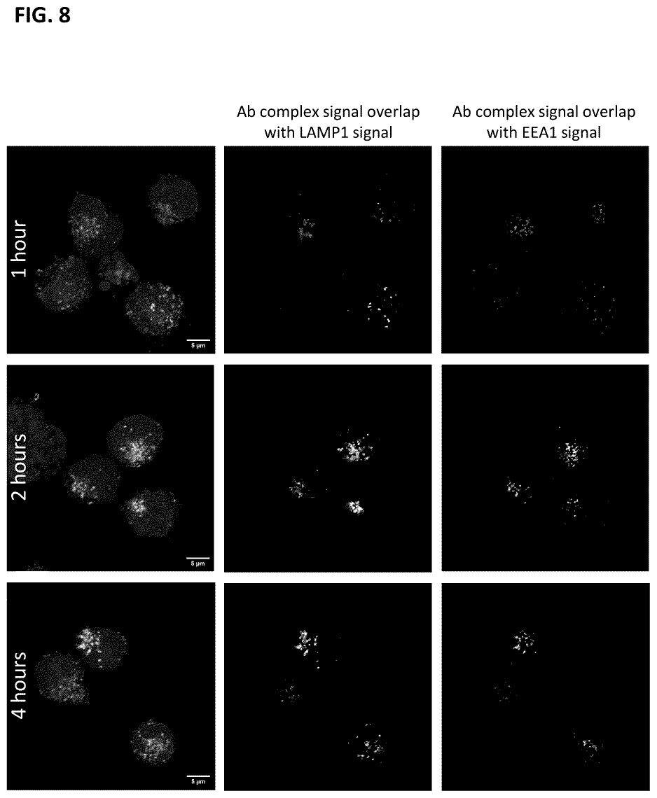

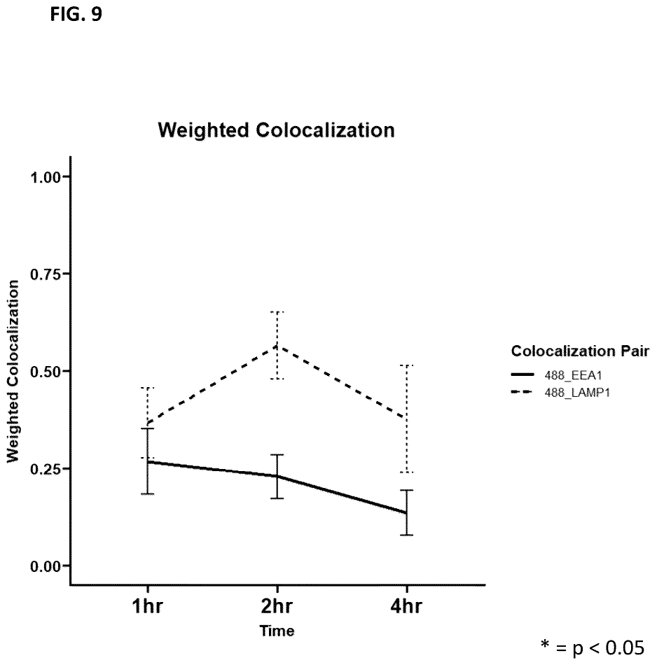

After incubation of the bispecific antibody complex with cells for 1-, 2-, or 4-hours (FIG. 6), samples were fixed and co-stained with endosome and lysosome markers using rabbit anti-EEA1 and mouse anti-LAMP1 antibodies, fluorescently-labeled goat anti-rabbit and goat anti-mouse secondary antibodies, as well as a nuclear marker (Hoechst) as shown in representative maximum intensity projection images (FIGS. 5 and 7). As shown in FIGS. 5-7, there was internalization of the bispecific antibody complex as early as 1-hour post incubation that overlapped with LAMP1 and/or EEA1 markers (FIG. 8, middle and right columns) which continued to accumulate at the 2- and 4-hour post incubation time point (FIGS. 5, 7, and 8). FIG. 6 confirms the accumulation of the bispecific antibody complex starting at the 1-hour post incubation time point that overlapped with LAMP1 and/or EEA1 markers. Most of the bispecific antibody complex colocalized with lysosomal marker, LAMP1, at the 2-hour incubation timepoint (FIG. 9). LAMP1 colocalization decreased at the 4-hour incubation timepoint potentially due to lysosomal degradation of the TNFα.

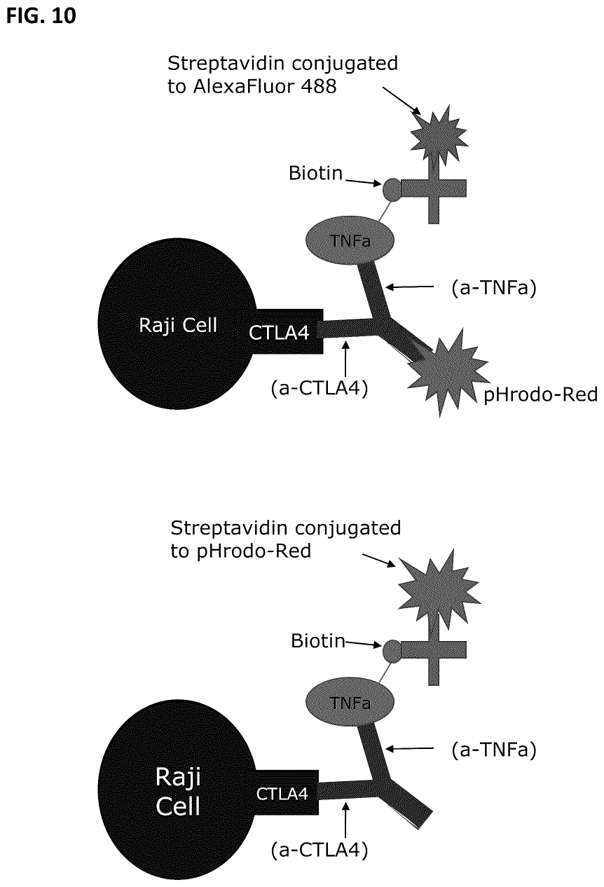

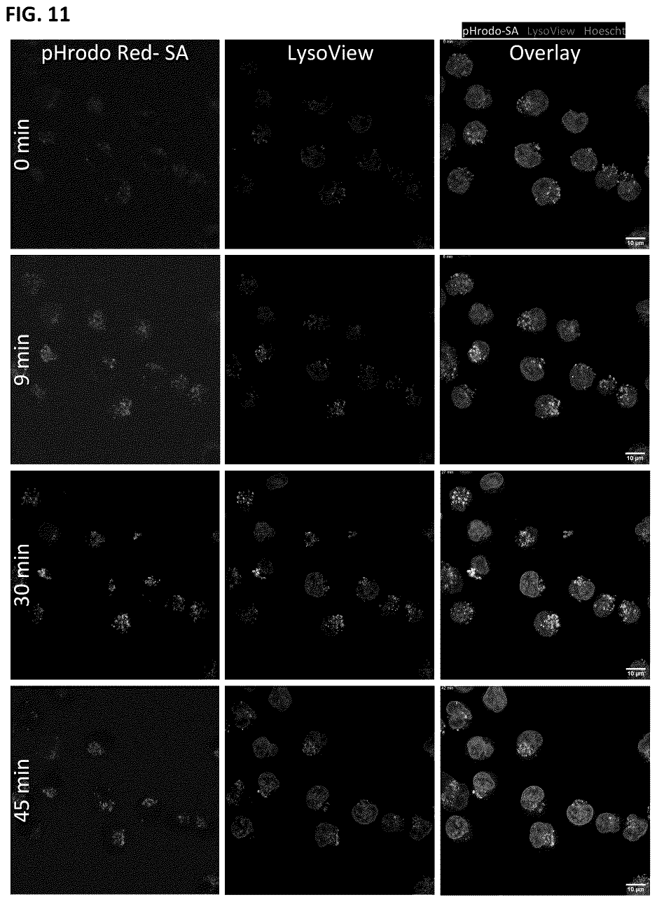

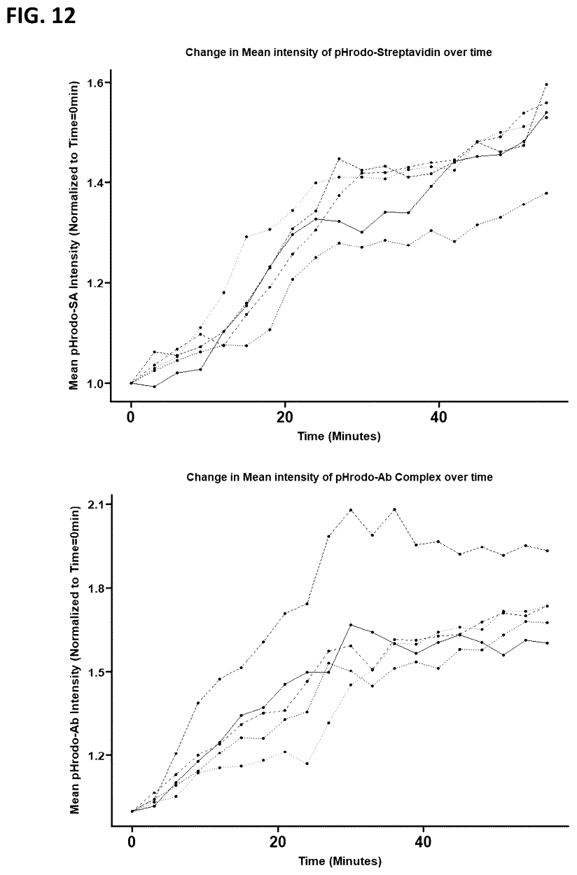

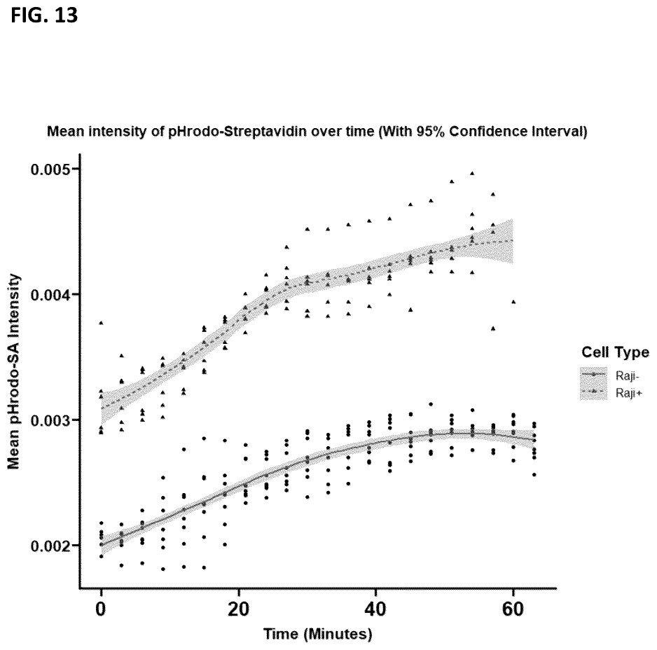

Unlike pH-insensitive fluorophores or pH-sensitive fluorophores that are brightly fluorescent at neutral pH, the fluorogenic nature of pHrodo red dye provides a ratiometric sensor for measuring the pH change in internal vesicular compartments like lysosomes and endosomes. Accordingly, the bispecific antibody complex described in FIG. 4 was modified by either conjugation of pHrodo-red to the bispecific antibody (FIG. 10 top) or to streptavidin-AF488 (FIG. 10 bottom) to confirm by a second method that the bispecific antibody complex was indeed taken up by Raji-CTLA-4 cells and shuttled into low pH internal compartments (e.g., lysosomes and endosomes). In fact, FIG. 11 confirms that the pHrodo-label and a lysosome stain (lysoview) overlap demonstrating that some of the low pH internal compartments were lysosomes. Supporting the fixed cell imaging, FIG. 12 confirms an increase in the intensity of the pHrodo label over time for at least the first hour of incubation. Regardless of whether the pHrodo-red was conjugated to the bispecific antibody or to the streptavidin-AF488 the mean pHrodo-red intensity increased over time after cell incubation with the bispecific antibody complex (FIG. 12). Further, this result was specific to the Raji-CTLA cell line relative to the control cells (Raji-null) as demonstrated by the difference in MFI intensity shown in FIG. 13.

Further supporting the microscopy data above, Raji-null or Raji-CTLA-4 cells were incubated with either 25 or 100 nM of the anti-TNFα/CTLA-4 bispecific antibody complex described in FIG. 4 or with isotype controls and analyzed via flow cytometric analysis. As shown in the FIG. 14B, there was only detection of intracellular stained TNFα in the Raji-CTLA-4 cells treated the anti-TNFα/CTLA-4 bispecific antibody complex as demonstrated by the specific CTLA-4-peak shift which was not observed in controls (see FIG. 14B bottom two panels).

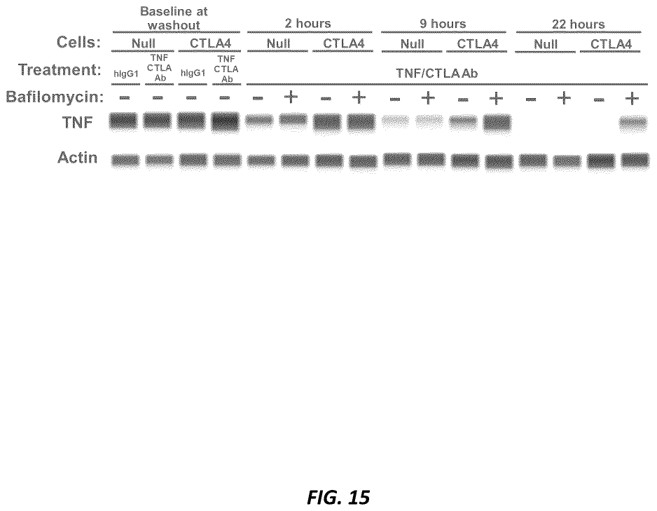

To biochemically confirm the results above, western blot analysis was performed. Raji-null or Raji-CTLA-4 cells were incubated with either the anti-TNFα/CTLA-4 bispecific antibody plus TNFα or with an antibody isotype control plus TNFα. As shown in FIG. 15, as early as the 9-hour time point TNFα was degraded in Raji-CTLA-4 cells which were incubated with the anti-TNFα/CTLA-4 bispecific antibody but not isotype controls. Further, when a potent lysosome inhibitor (bafilomycin) was applied to the samples TNFα was not degraded demonstrating that TNFα was being degraded via the lysosomal degradation pathway (see FIG. 15 at the 22-hour time point).

As shown in FIG. 16, cellular internalization could be recapitulated in phytohaemagglutinin (PHA) activated peripheral blood mononuclear cells (PBMC) from two different human donors. PBMC cells comprised about a 34.6% CD4⁺ T cell population as shown in the CD4⁺ gate in FIG. 16A. Upon incubation with TNFα and the anti-TNFα/CTLA-4 bispecific antibody activated PBMCs, which contain multiple T cell populations, internalize TNFα at both 4- and 24-hours post incubation (FIG. 16B).

In conclusion, the anti-TNFα/CTLA-4 bispecific antibody designed herein specifically bind to lymphocytes expressing surface CTLA-4 and the target protein (i.e., TNFα). Subsequently, the CTLA-4 surface expressing cell internalizes the anti-TNFα/CTLA-4 bispecific antibody bound to the TNFα and traffics in endosomal and lysosomal internal compartments via the lysosomal degradation pathway where TNFα is ultimately degraded. It is envisaged that the target protein binding arm of the multispecific binding proteins described herein can be modified to bind to a different target protein but maintain cell internalization utilizing cell surface expressing CTLA-4 and subsequent lysosomal trafficking pathway to degrade a target protein of interest.

### Example 3: Design of Anti-TNFα/CTLA-4 Bispecific Nanobody Constructs

To test whether other designs for multispecific binding proteins that bind to both TNFα and cell surface expressing CTLA-4 had the same potential to internalize and degrade TNFα, anti-TNFα/CTLA-4 bi-specific V_{HH} molecules were constructed.

The sequences for these constructs are shown in Table 3 and Table 4.

TABLE 3
Exemplary V_{HH} construct sequences

| SEQ ID | ISV sequence |
|---|---|
| Nb63 | EVQLVESGGGLVEPGGSLRLSCAASGSISSYNVMGWYRQAPGQQRDL |
| (SEQ ID | VAHIASNGEIMYADSAKGRFTISRDNAKKTVYLQMNSLKPEDTAVYY |
| NO: 7) | CKLWVLGNDYWGQGTLVTVSSGGGGSGGGGSGGGGSGGGGSGGGG |
| | SGGGGSGGGGSEVQLVESGGGVVQPGGSLRLSCAASGFTFSDYWMY |
| | WVRQAPGKGLEWVSEINTNGLITKYPDSVKGRFTISRDNAKNTLYLQ |
| | MNSLRPEDTALYYCARSPSGFNRGQGTLVTVSSGGGGSGGGGSGGGG |
| | SGGGGSGGGGSGGGGSGGGGSEVQLVESGGGVVQPGGSLRLSCAASG |
| | FTFSDYWMYWVRQAPGKGLEWVSEINTNGLITKYPDSVKGRFTISRDN |
| | AKNTLYLQMNSLRPEDTALYYCARSPSGFNRGQGTLVTVSSGGGGSG |
| | GGSEVQLVESGGGVVQPGGSLRLSCAASGFTFRSFGMSWVRQAPGKG |
| | PEWVSSISGSGSDTLYADSVKGRFTISRDNSKNTLYLQMNSLRPEDTAL |
| | YYCTIGGSLSRSSQGTLVTVSSA |
| Nb64 | EVQLVESGGGLVEPGGSLRLSCAASGSISSYNVMGWYRQAPGQQRDL |
| (SEQ ID | VAHIASNGEIMYADSAKGRFTISRDNAKKTVYLQMNSLKPEDTAVYY |
| NO: 8) | CKLWVLGNDYWGQRTLVTVSSGGGGSGGGGSGGGGSGGGGSGGGG |
| | SGGGGSGGGGSEVQLVESGGGVVQPGGSLRLSCAASGFTFSDYWMY |
| | WVRQAPGKGLEWVSEINTNGLITKYPDSVKGRFTISRDNAKNTLYLQ |
| | MNSLRPEDTALYYCARSPSGFNRGQGTLVTVSSGGGGSGGGGSGGGG |
| | SGGGGSGGGGSGGGGSGGGGSEVQLVESGGGVVQPGGSLRLSCAASG |
| | FTFSDYWMYWVRQAPGKGLEWVSEINTNGLITKYPDSVKGRFTISRDN |
| | AKNTLYLQMNSLRPEDTALYYCARSPSGFNRGQGTLVTVSSGGGGSG |
| | GGSEVQLVESGGGVVQPGGSLRLSCAASGFTFRSFGMSWVRQAPGKG |
| | PEWVSSISGSGSDTLYADSVKGRFTISRDNSKNTLYLQMNSLRPEDTAL |
| | YYCTIGGSLSRSSQGTLVTVSSA |
| Nb65 | EVQLVESGGGLVQPGGSLRLSCVASGIHFAISTINWYRQAPGKQRESV |
| (SEQ ID | AAITGTSVTGYADSVKGRFTLSRDNAKNTVYLQMDNLKPEDTAVYYC |
| NO: 9) | NVWSGRDYWGQGTLVTVSSGGGGSGGGGSGGGGSGGGGSGGGGSG |
| | GGGSGGGGSEVQLVESGGGVVQPGGSLRLSCAASGFTFSDYWMYWV |
| | RQAPGKGLEWVSEINTNGLITKYPDSVKGRFTISRDNAKNTLYLQMNS |
| | LRPEDTALYYCARSPSGFNRGQGTLVTVSSGGGGSGGGGSGGGGSGG |
| | GGSGGGGSGGGGSGGGGSEVQLVESGGGVVQPGGSLRLSCAASGFTF |
| | SDYWMYWVRQAPGKGLEWVSEINTNGLITKYPDSVKGRFTISRDNAK |
| | NTLYLQMNSLRPEDTALYYCARSPSGFNRGQGTLVTVSSGGGGSGGG |
| | SEVQLVESGGGVVQPGGSLRLSCAASGFTFRSFGMSWVRQAPGKGPE |
| | WVSSISGSGSDTLYADSVKGRFTISRDNSKNTLYLQMNSLRPEDTALY |
| | YCTIGGSLSRSSQGTLVTVSSA |

TABLE 4
Exemplary NANOBODY® fusion construct sequences

| SEQ ID | NANOBODY® sequence |
|---|---|
| CD80 | VIHVTKEVKEVATLSCGHNVSVEELAQTRIYWQKEKKMVLTMMSGD |
| (CD80-ECD)- | MNIWPEYKNRTIFDITNNLSIVILALRPSDEGTYECVVLKYEKDAFKRE |
| anti-TNF | HLAEVTLSVKADFPTPSISDFEIPTSNIRRIICSTSGGFPEPHLSWLENGEE |
| (SEQ ID | LNAINTTVSQDPETELYAVSSKLDFNMTTNHSFMCLIKYGHLRVNQTF |
| NO: 10) | NWNTTKQEHFPDNGGGGSGGGGSGGGGSGGGGSGGGGSGGGGSGGG |
| | GSEVQLVESGGGVVQPGGSLRLSCAASGFTFSDYWMYWVRQAPGKG |
| | LEWVSEINTNGLITKYPDSVKGRFTISRDNAKNTLYLQMNSLRPEDTAL |
| | YYCARSPSGFNRGQGTLVTVSSGGGGSGGGGSGGGGSGGGGSGGGGS |
| | GGGGSGGGGSEVQLVESGGGVVQPGGSLRLSCAASGFTFSDYWMYW |
| | VRQAPGKGLEWVSEINTNGLITKYPDSVKGRFTISRDNAKNTLYLQMN |
| | SLRPEDTALYYCARSPSGFNRGQGTLVTVSSGGGGSGGGSEVQLVESG |
| | GGVVQPGGSLRLSCAASGFTFRSFGMSWVRQAPGKGPEWVSSISGSGS |
| | DTLYADSVKGRFTISRDNSKNTLYLQMNSLRPEDTALYYCTIGGSLSRS |
| | SQGTLVTVSSA |
| CD86 | APLKIQAYFNETADLPCQFANSQNQSLSELVVFWQDQENLVLNEVYLG |
| (CD86-ECD)- | KEKFDSVHSKYMGRTSFDSDSWTLRLHNLQIKDKGLYQCIIHHKKPTG |
| anti-TNF | MIRIHQMNSELSVLANFSQPEIVPISNITENVYINLTCSSIHGYPEPKKMS |
| (SEQ ID | VLLRTKNSTIEYDGVMQKSQDNVTELYDVSISLSVSFPDVTSNMTIFCIL |
| NO: 11) | ETDKTRLLSSPFSIELEDPQPPPDHIPGGGGSGGGGSGGGGSGGGGSGG |
| | GGSGGGGSGGGGSEVQLVESGGGVVQPGGSLRLSCAASGFTFSDYWM |
| | YWVRQAPGKGLEWVSEINTNGLITKYPDSVKGRFTISRDNAKNTLYLQ |
| | MNSLRPEDTALYYCARSPSGFNRGQGTLVTVSSGGGGSGGGGSGGGG |
| | SGGGGSGGGGSGGGGSGGGGSEVQLVESGGGVVQPGGSLRLSCAASG |
| | FTFSDYWMYWVRQAPGKGLEWVSEINTNGLITKYPDSVKGRFTISRDN |
| | AKNTLYLQMNSLRPEDTALYYCARSPSGFNRGQGTLVTVSSGGGGSG |
| | GGSEVQLVESGGGVVQPGGSLRLSCAASGFTFRSFGMSWVRQAPGKG |
| | PEWVSSISGSGSDTLYADSVKGRFTISRDNSKNTLYLQMNSLRPEDTAL |
| | YYCTIGGSLSRSSQGTLVTVSSA |

### Example 4: Cellular Internalization and Degradation of TNFα Via an Anti-TNFα/CTLA-4 Bispecific Antibody

Using the methods described in Example 2, the constructs made in Example 3 were tested for their ability facilitate cellular internalization in a CTLA-4 cell surface expressing cell and subsequently degraded TNFα via the lysosomal degradation pathway.

### Methods

### TNFα Internalization Assay by Flow Cytometry

Raji-null and Raji-CTLA-4 cells were plated at a concentration of 10⁵ cells/well in U-bottom 96 well plates. Recombinant biotinylated TNFα at was first added to the 96-well plate at a final concentration of 50 nM, streptavidin-Alexa647 was then added to the 96-well plate at a final concentration of 100 nM, and either anti-TNFα V_{HH}, anti-CTLA-4 V_{HH}s, or anti-TNFα/CTLA-4 bi-specific V_{HH} constructs were finally added to the 96-well plate at a final concentration of 25 or 100 nM. After the cells were cultured at 37° C. for 4 hours, they were washed twice with cold phosphate buffered saline (PBS), then flow cytometry was conducted to assess Alexa647 fluorescence.

### TNFα Degradation Assay by Western Blotting

Raji-CTLA4 cells were plated at a concentration of 10⁵ cells/well in U-bottom 96 well plates. Recombinant TNFα was then added to 96-well plate at a final concentration of 50 nM, and either anti-TNFα V_{HH}, anti-CTLA-4 V_{HH}, or anti-TNFα/CTLA-4 bi-specific V_{HH} constructs were then added to 96-well plate at a final concentration of 25 or 100 nM. After the cells were cultured at 37° C. for 2 hours, they were washed twice with media and lysate was produced using radioimmunoprecipitation assay (RIPA) buffer. A few cells remained in culture for an additional 2, 9, 24 hours in the presence of DMSO or 100 nM Bafilomycin A. At each time point, the cells were washed twice with cold PBS then RIPA buffer to produce a cell lysate. Western blotting was employed to detect TNFα or β-actin with the prepared lysate.

In another experiment, Raji-CTLA-4 cells were plated at a concentration of 10⁵ cells well in U-bottom 96 well plates. Recombinant TNF was then added to the 96-well plate at a final concentration of 12.5 or 50 nM, and either anti-TNFα V_{HH}, anti-CTLA-4 V_{HH}s, or anti-TNFα/CTLA-4 bi-specific V_{HH} constructs were then added to 96-well plate at a final concentration of 25 or 100 nM. After the cells were cultured at 37° C. for 24, 48, and 72 hours, the cell culture media was collected and analyzed using western blotting.

### Results

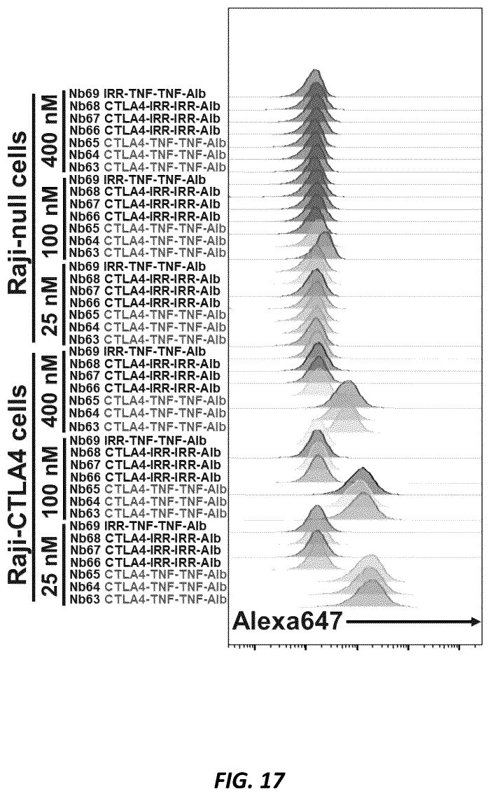

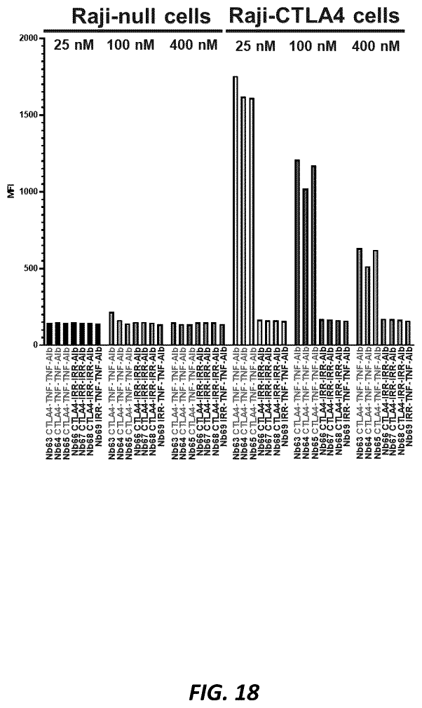

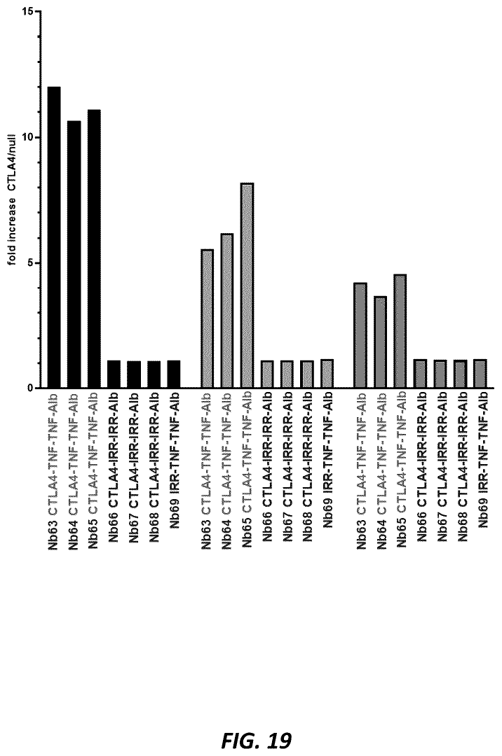

As shown in FIG. 17, TNFα internalization was observed in Raji-CTLA-4 cells upon incubation with TNFα and 25, 100, or 400 nM of anti-TNFα/CTLA-4 bi-specific V_{HH} constructs designed in Example 3 as demonstrated by the MFI peak shift which was not observed in incubation with control V_{HH} constructs or with Raji-null cells. A graphical representation of the results in FIG. 17 are shown in FIG. 18. FIG. 19 displays the fold increase of TNFα internalization by anti-TNF/CTLA-4 V_{HH} constructs or V_{HH} control constructs in Raji-CTLA-4 cells relative to Raji-null cells. The results of FIG. 19 demonstrate that specificity for binding both TNFα and cell surface expressing CTLA-4 are required for TNFα internalization.

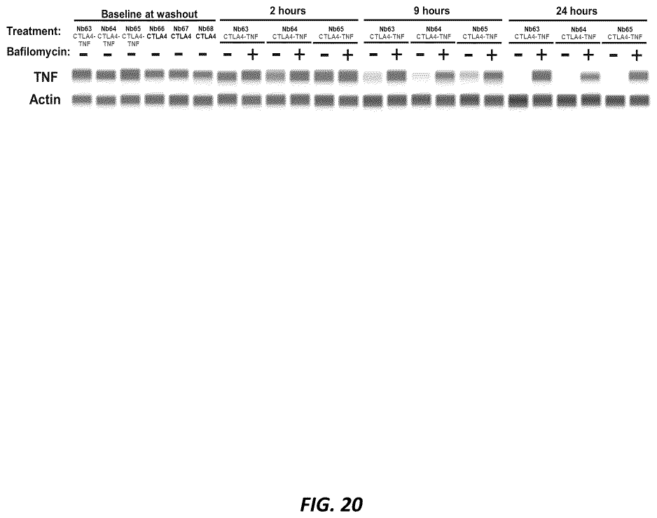

To confirm TNFα was degraded via the lysosomal pathway upon cell internalization western blot analysis was performed. As shown in FIG. 20, TNFα was degraded when Raji-CTLA-4 cells had been incubated with TNFα plus the anti-TNFα/CTLA-4 V_{HH} constructs as early as the 9-hour post incubation timepoint. When bafilomycin was applied the samples TNFα was not degraded demonstrating that TNFα was degraded via the lysosomal degradation pathway (see FIG. 20 at the 9- and 24-hour time point).

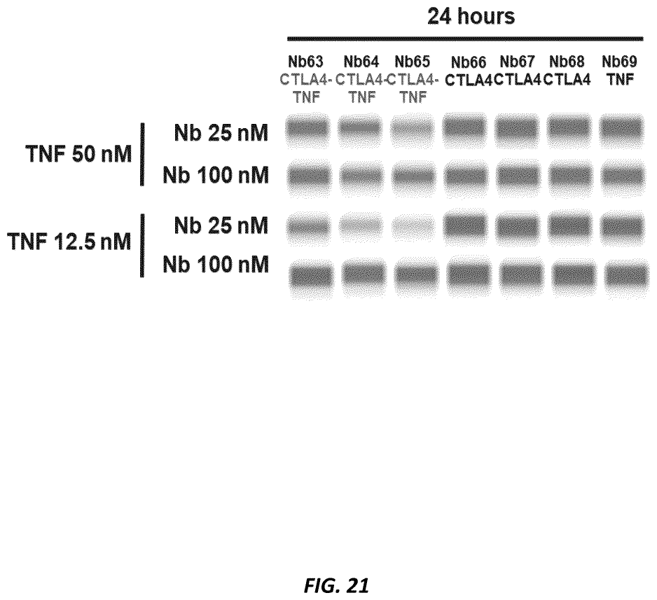

To show that soluble TNFα was reduced from the medium upon a 24-hour incubation of Raji-CTLA-4 cells with TNFα plus the anti-TNFα/CTLA-4 V_{HH} constructs or controls, TNFα was added at two different concentrations (12.5 and 50 nM). As shown in FIG. 21, TNFα was reduced in the cell medium of the cells incubated with anti-TNFα/CTLA-4 V_{HH} constructs but not with the controls.

In conclusion, other multispecific binding protein construct designs, such as the anti-TNFα/CTLA-4 V_{HH} constructs described herein can facilitate target protein (e.g., TNFα) internalization and degradation by binding to a surface expressing CTLA-4 cell. Upon binding and internalization, the TNF/CTLA-4 V_{HH} constructs plus the target protein are trafficked and degraded via the lysosomal pathway. Accordingly, various binding protein formats can be utilized to engineer the CTLA-4 multispecific binding proteins described herein if the binding arm as long as the cell surface expressing CTLA-4 binding arm is maintained. As discussed above, any target protein of interest can be engineered into the multispecific binding protein constructs described herein.

1. A method for degrading a target protein, comprising:
contacting a T-cell with a multispecific binding protein, wherein the multispecific binding protein comprises:
a) a first cell surface binding moiety that specifically binds to membrane-bound CTLA-4 on the surface of the T-cell; and
b) a second binding moiety that is operatively linked to the first cell surface binding moiety and that specifically binds to the target protein,
wherein binding of the multispecific binding protein to the membrane-bound CTLA-4 on the surface of the T-cell facilitates the internalization of the target protein by the T-cell.

2. The method of claim 1, wherein after internalization the target protein is degraded in a lysosome.

3-7. (canceled)

8. The method of claim 1, wherein the first cell surface binding moiety comprises a CD80-fragment crystallizable (Fc) fusion polypeptide or a CD86-Fc fusion polypeptide.

9. The method of claim 1, wherein the target protein is selected from the group consisting of an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

10-17. (canceled)

18. The method of claim 1, wherein the first cell surface binding moiety of the multispecific binding protein that specifically binds to membrane-bound CTLA-4 on the surface of the T-cell is an antibody or an antigen binding fragment thereof, an Fc fusion protein, a fragment antigen-binding (Fab) fusion, an immunoglobulin single variable domain (ISV), or a single-chain fragment variable (scFv).

19-23. (canceled)

24. The method of claim 1, wherein the second binding moiety of the multispecific binding protein that specifically binds to the target protein is selected from the group consisting of an antigen, an antibody or an antigen binding fragment thereof, an autoantibody, a Fc fusion protein, a Fab, an scFv, an ISV, an AFFIBODY®, or a peptide.

25-27. (canceled)

28. The method of claim 1, wherein the first cell surface binding moiety of the multispecific binding protein exhibits pH-dependent binding to membrane-bound CTLA-4 on the surface of the T-cell.

29-30. (canceled)

31. The method of claim 1, wherein the first cell surface binding moiety or the second binding moiety of the multispecific binding protein binds to membrane-bound CTLA-4 on the surface of the T-cell with an affinity from about 100 pM to about 1 μM.

32. (canceled)

33. The method of claim 1, wherein the multispecific binding protein comprises one or more mutations or glycan modifications to modulate the Fc mediated effector function and/or the multispecific binding protein comprises one or more mutations to modulate half-life.

34. (canceled)

35. A multispecific binding protein comprising:
a) a first cell surface binding moiety that specifically binds to membrane-bound CTLA-4 on the surface of a T-cell; and
b) a second binding moiety that is operatively linked to the first cell surface binding moiety and that specifically binds to a target protein,
such that the multispecific binding protein binds to the membrane-bound CTLA-4 on the surface of the T-cell and to the target protein,
wherein binding to the membrane-bound CTLA-4 on the surface of the T-cell facilitates the internalization of the target protein by the T-cell.

36. The multispecific binding protein claim 35, wherein after internalization the target protein is degraded in a lysosome.

37. (canceled)

38. The multispecific binding protein of claim 35, wherein the first cell surface binding moiety that specifically binds to membrane-bound CTLA-4 on the surface of the T-cell is a CTLA-4 ligand.

39. (canceled)

40. The multispecific binding protein of claim 35, wherein the target protein is selected from the group consisting of an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

41-48. (canceled)

49. The multispecific binding protein of claim 35, wherein the first cell surface binding moiety of the multispecific binding protein that specifically binds to membrane-bound CTLA-4 on the surface of the T-cell is an antibody or an antigen binding fragment thereof, an Fc fusion protein, a Fab fusion, an ISV, or a scFv.

50-54. (canceled)

55. The multispecific binding protein of claim 35, wherein the second binding moiety that specifically binds to the target protein is selected from the group consisting of an antigen, an antibody or an antigen binding fragment thereof, an autoantibody, a Fc fusion, a Fab, an scFv, an ISV, an AFFIBODY®, or a peptide.

56-65. (canceled)

66. A pharmaceutical composition comprising the multispecific binding protein of claim 35 and a pharmaceutically acceptable carrier.

67. An isolated nucleic acid molecule encoding a multispecific binding protein of comprising:
a) a first cell surface binding moiety that specifically binds to membrane-bound CTLA-4 on the surface of a T-cell; and
b) a second binding moiety that is operatively linked to the first cell surface binding moiety and that specifically binds to a target protein,
such that the multispecific binding protein binds to the membrane-bound CTLA-4 on the surface of the T-cell and to the target protein,
wherein binding to the membrane-bound CTLA-4 on the surface of the T-cell facilitates the internalization of the target protein by the T-cell.

68. An expression vector or a host cell comprising the expression vector comprising the nucleic acid molecule of claim 67.

69. (canceled)

70. A method of treating a subject with a disease associated with the target protein or soluble target protein, comprising administering to the subject a therapeutically effective amount of a multispecific binding protein comprising:
a) a first cell surface binding moiety that specifically binds to membrane-bound CTLA-4 on the surface of a T-cell; and
b) a second binding moiety that is operatively linked to the first cell surface binding moiety and that specifically binds to a target protein,
such that the multispecific binding protein binds to the membrane-bound CTLA-4 on the surface of the T-cell and to the target protein,
wherein binding to the membrane-bound CTLA-4 on the surface of the T-cell facilitates the internalization of the target protein by the T-cell.

71-76. (canceled)

77. A method of making a multispecific binding protein, the method comprising expressing the isolated nucleic acid molecule of claim 67 in an expression vector introduced into a host cell under conditions sufficient to produce the multispecific binding protein.


SEQUENCE LISTING (58 sequences)

SEQ ID NO: 1 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 447 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 5 - protein, 439 aa, synthetic construct
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPG

SEQ ID NO: 6 - protein, 455 aa, synthetic construct
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPG

SEQ ID NO: 7 - protein, 540 aa, synthetic construct
EVQLVESGGGLVEPGGSLRLSCAASGSISSYNVMGWYRQAPGQQRDLVAHIASNGEIMYADSAKGRFTISRDNAKKTVYLQMNSLKPEDTAVYYCKLWVLGNDYWGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 8 - protein, 540 aa, synthetic construct
EVQLVESGGGLVEPGGSLRLSCAASGSISSYNVMGWYRQAPGQQRDLVAHIASNGEIMYADSAKGRFTISRDNAKKTVYLQMNSLKPEDTAVYYCKLWVLGNDYWGQRTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 9 - protein, 539 aa, synthetic construct
EVQLVESGGGLVQPGGSLRLSCVASGIHFAISTINWYRQAPGKQRESVAAITGTSVTGYADSVKGRFTLSRDNAKNTVYLQMDNLKPEDTAVYYCNVWSGRDYWGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 10 - protein, 633 aa, synthetic construct
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 11 - protein, 649 aa, synthetic construct
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGGSGGGGSGGGGSGGGGSGGGGSGGGGSEVQLVESGGGVVQPGGSLRLSCAASGFTFSDYWMYWVRQAPGKGLEWVSEINTNGLITKYPDSVKGRFTISRDNAKNTLYLQMNSLRPEDTALYYCARSPSGFNRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 12 - protein, 4 aa, synthetic construct
KERE

SEQ ID NO: 13 - protein, 4 aa, synthetic construct
KQRE

SEQ ID NO: 14 - protein, 5 aa, synthetic construct
KEREL

SEQ ID NO: 15 - protein, 5 aa, synthetic construct
KEREF

SEQ ID NO: 16 - protein, 5 aa, synthetic construct
KQREL

SEQ ID NO: 17 - protein, 5 aa, synthetic construct
KQREF

SEQ ID NO: 18 - protein, 5 aa, synthetic construct
KEREG

SEQ ID NO: 19 - protein, 5 aa, synthetic construct
KQREW

SEQ ID NO: 20 - protein, 5 aa, synthetic construct
KQREG

SEQ ID NO: 21 - protein, 4 aa, synthetic construct
TERE

SEQ ID NO: 22 - protein, 5 aa, synthetic construct
TEREL

SEQ ID NO: 23 - protein, 4 aa, synthetic construct
TQRE

SEQ ID NO: 24 - protein, 5 aa, synthetic construct
TQREL

SEQ ID NO: 25 - protein, 4 aa, synthetic construct
KECE

SEQ ID NO: 26 - protein, 5 aa, synthetic construct
KECEL

SEQ ID NO: 27 - protein, 5 aa, synthetic construct
KECER

SEQ ID NO: 28 - protein, 4 aa, synthetic construct
KQCE

SEQ ID NO: 29 - protein, 5 aa, synthetic construct
KQCEL

SEQ ID NO: 30 - protein, 4 aa, synthetic construct
RERE

SEQ ID NO: 31 - protein, 5 aa, synthetic construct
REREG

SEQ ID NO: 32 - protein, 5 aa, synthetic construct
RQREL

SEQ ID NO: 33 - protein, 5 aa, synthetic construct
RQREF

SEQ ID NO: 34 - protein, 5 aa, synthetic construct
RQREW

SEQ ID NO: 35 - protein, 4 aa, synthetic construct
QERE

SEQ ID NO: 36 - protein, 5 aa, synthetic construct
QEREG

SEQ ID NO: 37 - protein, 4 aa, synthetic construct
QQRE

SEQ ID NO: 38 - protein, 5 aa, synthetic construct
QQREW

SEQ ID NO: 39 - protein, 5 aa, synthetic construct
QQREL

SEQ ID NO: 40 - protein, 5 aa, synthetic construct
QQREF

SEQ ID NO: 41 - protein, 4 aa, synthetic construct
KGRE

SEQ ID NO: 42 - protein, 5 aa, synthetic construct
KGREG

SEQ ID NO: 43 - protein, 4 aa, synthetic construct
KDRE

SEQ ID NO: 44 - protein, 5 aa, synthetic construct
KDREV

SEQ ID NO: 45 - protein, 5 aa, synthetic construct
DECKL

SEQ ID NO: 46 - protein, 5 aa, synthetic construct
NVCEL

SEQ ID NO: 47 - protein, 4 aa, synthetic construct
GLEW

SEQ ID NO: 48 - protein, 4 aa, synthetic construct
GVEW

SEQ ID NO: 49 - protein, 4 aa, synthetic construct
EPEW

SEQ ID NO: 50 - protein, 4 aa, synthetic construct
GLER

SEQ ID NO: 51 - protein, 4 aa, synthetic construct
DQEW

SEQ ID NO: 52 - protein, 4 aa, synthetic construct
DLEW

SEQ ID NO: 53 - protein, 4 aa, synthetic construct
GIEW

SEQ ID NO: 54 - protein, 4 aa, synthetic construct
ELEW

SEQ ID NO: 55 - protein, 4 aa, synthetic construct
GPEW

SEQ ID NO: 56 - protein, 4 aa, synthetic construct
EWLP

SEQ ID NO: 57 - protein, 4 aa, synthetic construct
GPER

SEQ ID NO: 58 - protein, 4 aa, synthetic construct
RQRE